(19) United States
(12) Patent Application Publication
(10) Pub. No.: US 2004/0176721 A1
(43) Pub. Date: Sep. 9, 2004

(54) Needleless hypodermic injection device with non-electric primer ignition means

(76) Inventors: Otto Furst, Viernheim (DE); Hans-Peter Haar, Wiesloch (DE); George Bevan Kirby Meacham, Shaker Heights, OH (US); Hans List, Hesseneck (DE)

Correspondence Address: HOFFMANN-LA ROCHE INC., PATENT LAW DEPARTMENT, 340 KINGSLAND STREET, NUTLEY, NJ, 07110

(21) Appl. No.: 10/776,974

(22) Filed: Feb. 11, 2004

Related U.S. Application Data
(60) Provisional application No. 60/449,820, filed on Feb. 25, 2003.

(51) Int. Cl. A61M 5/30

(52) U.S. Cl. 604/70

(57) ABSTRACT

A single use needleless hypodermic injection device for injecting a liquid medication contained therein. The device includes pyrotechnical means for generating within the device a pressure necessary for injecting the medication. The device comprises impact-sensitive stab primer ignition means for igniting a propellant contained in the device. The stab primers ignite with less that 10 millijoules of mechanical energy, enabling small, low cost firing pin mechanisms contained within the device that may be economically discarded after a single use

### PRIORITY TO PROVISIONAL APPLICATION(s) UNDER 35 U.S.C. §119(e)

This application claims priority under 35 U.S.C.§119(e) of provisional application(s) Serial No. 60/449,820, filed on Feb. 25, 2003.

### FIELD OF THE INVENTION

The invention concerns a needleless hypodermic injection device for delivering liquid medication contained in the device. The device includes pyrotechnical means therein for generating a pressure necessary for injecting the medication. An ignition means ignites a propellant contained withinin the device.

### BACKGROUND OF THE INVENTION

U.S. Pat. No. 6,258,063 discloses a needleless hypodermic injection device comprising electric ignition means for igniting a propellant contained in the device and thereby generating the gas pressure necessary for performing the injection. Electric ignition requires the use of batteries as source of energy. Use of batteries is disadvantageous, because disposal of batteries in normal trash is not allowed due to environmental concerns. Thus electric ignition is not a suitable solution for a needleless injection device that should be disposable in normal trash after a single or at most a few injections performed therewith. Moreover, it is wasteful to discard the entire injection device with its battery after a single use, because the battery could still be used for performing more than a single injection.

Heavy metal-free impact sensitive pyrotechnic ignition primers provide an environmentally acceptable means of igniting propellant. Typically, a cocked spring and firing pin are released by a trigger to strike and ignite impact sensitive pyrotechnic ignition material. The heat and products of combustion of the ignition material in turn ignite the propellant. A variety of such primers is known in the art, and may be purchased or manufactured at low cost.

A problem with utilizing such primers in a single use disposable device is the need to provide a firing pin, spring and trigger mechanism that functions reliably, yet has low manufacturing cost.

Another problem with utilizing such primers is the integration of the primer and the firing pin, spring and trigger mechanism with the injection device such that the total system is structurally sound, leak-tight and compact. The magnitude of these problems increases as the firing pin mechanical energy requirement increases.

### SUMMARY OF THE INVENTION

The invention herein solves the above-mentioned problems and thereby to reduce the manufacturing cost of the injection device and to maintain and preferably even increase the reliability of operation of the latter device.

The invention concerns in particular a device of the above mentioned kind wherein the device comprises a stab primer device as part of the ignition means.

Within the scope of the instant invention, a propellant is a pyrotechnic fuel which mainly contributes to the delivery of thermal energy and gas production of a pyrotechnic system.

Within the scope of the instant invention, a stab primer ignition means is an assembly that contains at least a sensitive primary pyrotechnic initiator material that is mechanically ignited by the direct impact and friction of a penetrating firing pin. It may also contain secondary pyrotechnic ignition materials and propellant that are ignited in a chain of events started by the ignition of the primary pyrotechnic initiator material. Further, it may be part of a unitary package that includes an outer container and seals that protect the pyrotechnic materials from external effects such as atmospheric moisture.

The above-mentioned problems are solved by using a primer device comprising a primer material which is adapted to be ignited by friction of the primer material with a mechanical frictional element. In a preferred embodiment, the frictional element is an elongated rod a portion of which is embedded in the primer material and the outer surface of the portion has serrations that cause frictional forces when the rod portion is pulled out of the primer material. In preferred embodiments, the primer device used is a stab primer device.

The main advantages of a device according to the invention are obtained by the use of primer devices of the above mentioned kind and in particular by the use of stab primer devices as part of the ignition means, because activation of stab primers requires very low mechanical energy. They typically fire reliably with less than 10 millijoules firing pin energy. In comparison, primers used in centerfire pistol ammunition require 150 millijoules.

Commercially manufactured stab primers are cylindrical assemblies ranging from 2 to 6 millimeters in diameter and 4 to 12 millimeters in length. There are two basic configurations available. The outer package of the first is a metallic cup. These one-side-open igniters are struck and ignited by a firing pin traveling along the cylindrical axis and entering the open end of the cup. The products of combustion then flow out through the open cup end around the firing pin, while the other end remains sealed. The outer package of the second is a metallic tube. These two-side-open igniters are struck and ignited by a firing pin traveling along the cylindrical axis and entering one open end of the tube. The products of combustion then flow out of both open ends.

In preferred embodiments in which the primer device is a stab primer device, the latter device contains a firing pin and a bistable spring for driving the firing pin so that it penetrates a primer material contained in the stab primer device.

According to a first aspect of the invention, a needleless hypodermic injection device for delivering a liquid medication contained therein includes pyrotechnical means for generating within the device a pressure necessary for injecting the medication and the device comprises ignition means for igniting a propellant contained in the device. The device further comprises a stab primer device and a firing pin for penetrating a stab primer material stationarily arranged within the stab primer device. The stab primer material is so positioned with respect to the propellant that when the firing pin penetrates the primer material, hot products of combustion of the primer material are generated and these products ignite the propellant.

In a preferred embodiment, the latter device further comprises a spring for urging the firing pin towards the primer material, and a release latch for holding the spring in a loaded position and thereby the firing pin in a cocked position and for releasing the spring and thereby drive the firing pin towards the primer material. The stab primer device preferably comprises a stab primer open on two sides opposite to each other.

According to a second aspect of the invention, a needleless hypodermic injection device for delivering a liquid medication contained therein includes pyrotechnical means for generating within the device a pressure necessary for injecting the medication and the device comprises ignition means for igniting a propellant contained in the device. The device further comprises

a slidably mounted stab primer device which is open on only one side and with the open side arranged in face of a sharp point of a stationary stab pin,

an impact plunger for driving the stab primer device towards the stab pin so that the pin penetrates into a primer material contained in the stab primer device,

a spring for urging the plunger towards the primer material, and

a release latch for releasably holding the spring and thereby the plunger in a cocked position.

In a first preferred embodiment, the device disclosed immediately above further comprises release means for releasing the release latch. The release means preferably comprise a breakable crimp joint or a breakable rod.

According to a third aspect of the invention, in a preferred embodiment of the device according to the above-mentioned second aspect of the invention, the impact plunger comprises a tapered section and a hook for setting the plunger in a cocked position, and the release latch is a release lever for releasing the hook and for thereby releasing the impact plunger from the cocked position.

According to a fourth aspect of the invention, a needleless hypodermic injection device for delivering liquid medication contained therein includes pyrotechnical means for generating within the device a pressure necessary for injecting the medication and the device comprises ignition means for igniting a propellant contained in the device. The device further comprises a stab primer device and a firing pin for penetrating a stab primer material stationarily arranged within the stab primer device. The stab primer material is so positioned with respect to the propellant that when the firing pin penetrates the primer material, hot products of combustion of the primer material are generated and these products ignite the propellant. The device further comprises a bistable spring for urging the firing pin towards the primer material, the bistable spring being adapted to snap at a transition point from a first stable position to a second stable position. In a first preferred embodiment of the latter device the bistable spring and the firing pin are integral part of the structure of the stab primer.

In a second preferred embodiment of the device disclosed immediately above, it further comprises an actuation screw which when turned in a predetermined position pushes the bistable spring and the firing pin towards the primer material and thereby brings the spring to the transition point where it snaps from the first to the second position, the latter snapping causing the firing pin to penetrate and ignite the primer material.

According to a fifth aspect of the invention, in a third preferred embodiment of the device according to the above-mentioned fourth aspect of the invention, the device further comprises an actuation push pin which when axially displaced in a predetermined position, pushes the bistable spring and the firing pin towards the primer material and thereby brings the spring to the transition point where it snaps from the first to the second position, the latter snapping causes the firing pin to penetrate and ignite the primer. The bistable spring preferably seals an opening of the stab primer device.

In a preferred embodiment of the device according to the fifth aspect of the invention, the device further comprises a venting passage which fluidically connects spaces on opposite sides of the bistable spring and thereby enables gas flow around the bistable spring. The bistable spring has preferably the shape of a disk. In a preferred embodiment the bistable spring comprises vents that equalize pressure on both sides of the disk. In another preferred embodiment the ignition means is an integral part of a pre-assembled gas generator module.

According to a sixth aspect of the invention, a needleless hypodermic injection device for delivering liquid medication contained therein includes pyrotechnical means for generating within the device a pressure necessary for injecting the medication and the device comprises ignition means for igniting a propellant contained in the device. The device further comprises a stab primer device and a firing pin for penetrating a stab primer material stationarily arranged within the stab primer device. The stab primer material is so positioned with respect to the propellant that when the firing pin penetrates the primer material, hot products of combustion of the primer material are generated and these products ignite the propellant. The firing pin ends in a firing pin head located within a closed chamber, which seals a space of limited volume located between the firing pin head and one side of the stab primer.

According to a seventh aspect of the invention, a needleless hypodermic injection device for delivering liquid medication contained therein includes pyrotechnical means for generating within the device a pressure necessary for injecting the medication and the device comprises ignition means for igniting a propellant contained in the device. The device further comprises a stab primer device and a firing pin for penetrating a stab primer material stationarily arranged within the stab primer device. The stab primer material is so positioned with respect to the propellant that when the firing pin penetrates the primer material, hot products of combustion of the primer material are generated and these products ignite the propellant. The device further comprises a spring for urging the firing pin towards the primer material, and a release mechanism for holding the spring in a loaded position, and thereby the firing pin in a cocked position and for releasing the spring and thereby drive the firing pin towards the primer material.

In a preferred embodiment of the latter device the firing pin, the spring and the release mechanism are located in a closed space into which hot gases generated by ignition of the primer material and the propellant flow.

In a further preferred embodiment, the release mechanism comprises means for rotating the firing pin using torque applied by means which is located outside the device. This rotation brings the firing pin from a first angular position where it is in a cocked position to a second angular position where the pin is free to move axially and making contact with the primer material.

According to a eighth aspect of the invention, in a preferred embodiment of the device according to the above-mentioned seventh aspect of the invention, the release mechanism of the device comprises a shaft adapted to be twisted from a first angular position to a second angular position for releasing the spring. The shaft is in contact with the firing pin in the cocked position thereof, but is mechanically disconnected therefrom so that when the firing pin is released from its cocked position and moves towards the primer material, the shaft does not move with the firing pin. In a preferred embodiment, the shaft includes flange means, which seal an annular opening around the shaft when pressure pushes the shaft to the rear of the device. In a further preferred embodiment, the ignition means is an integral part of a preassembled gas generator module.

According to a ninth aspect of the invention, a needleless hypodermic injection device for delivering liquid medication contained therein comprises

(a) a cartridge which contains

(a.1) a medication unit containing the liquid medication,

(a.2) pyrotechnical means for generating within the device a pressure necessary for injecting the medication, and

(a.3) ignition means for igniting a propellant contained in the device, and

(b) a spring and trigger mechanism for striking the firing pin with such an impact that it strikes and penetrates the primer material.

The spring and trigger mechanism are located outside the cartridge. The ignition means comprises a stab primer device and a firing pin for penetrating a stab primer material stationarily arranged within the device. The stab primer material is so positioned with respect to the propellant that when the firing pin penetrates the primer material, hot products of combustion of the primer material are generated and these products ignite the propellant.

In a preferred embodiment, the firing pin is slidably arranged in a bore of a housing part of the cartridge. A portion of the inner wall of the bore has ratchet fingers. A part of the firing pin is a shaft a portion of which has a ratchet grooves. The ratchet fingers and the ratchet grooves are adapted to cooperate with each other to allow motion of the firing pin towards the primer material, but to prevent motion of the firing pin away from the primer material after ignition thereof.

In a preferred embodiment, the ignition means is an integral part of a preassembled gas generator module.

In all the above mentioned embodiments comprising a stab primer device, the entire amount of propellant in the device is preferably located within the stab primer device. However, for particular applications the device may comprise an amount of propellant located outside of the stab primer device.

Any and all the above mentioned embodiments are suitable for being used as part of a first type of device which comprises

(a) a housing,

(b) a first chamber within the housing, the first chamber containing a medication unit configured and dimensioned to store a volume of liquid medication to be injected, the medication unit having a first region and a second region that are in liquid communication with each other, the first region being deformable and the second region having an ejection outlet, and

(c) a second chamber within the housing, the second chamber containing a propellant, the first chamber comprising two zones, a first zone containing the medication unit and a second zone which is in communication with the second chamber, so that upon ignition of the propellant in the second chamber gas generated thereby expands into the second zone of the first chamber, exerts pressure on and deforms the deformable first region of the medication unit and thereby causes ejection of the liquid medication through the ejection outlet.

In a preferred embodiment of this first type of device, the propellant is contained in a propellant chamber having a wall which has a zone of reduced thickness which upon ignition of the propellant bursts and thereby forms an opening of the wall when gas pressure within the propellant chamber exceeds a predetermined value.

Any and all the above mentioned embodiments are suitable for being used as part of a second type of device which comprises a nozzle body, and a rigid housing. The housing has a first open end adapted to receive and be connected with the nozzle body and a second closed end. The interior of the housing defines a chamber which extends between the open end and the closed end of the housing. The chamber is adapted to receive a first deformable diaphragm which together with a cavity of the nozzle body forms a medication chamber suitable for receiving a predetermined amount of a medication, and a second deformable diaphragm a portion of which extends around a portion of the first deformable diaphragm. The second deformable diaphragm and the housing form together a chamber for receiving a propellant as well as means for igniting the propellant. The nozzle body has at its outer end an orifice which is the outlet of a channel for ejecting the medication out of the chamber when a gas pressure generated by ignition of the propellant is applied to the second deformable diaphragm and thereby to the first deformable diaphragm.

In a preferred embodiment, the nozzle body and the housing are connected to form a single structural shell.

In another preferred embodiment, the device further comprises venting means for venting of the space comprised between the first deformable diaphragm and the second deformable diaphragm.

In a further preferred embodiment, the device does not include the second deformable diaphragm.

Any and all the above mentioned embodiments are suitable for being used as part of a third type of device which comprises

(a) a rigid medication container having a medication zone for receiving the liquid medication,

(b) a nozzle in fluidic communication with the medication zone, the nozzle having an outlet orifice,

(c) a propellant zone where the propellant is located within the device,

(d) a channel that fluidically connects the propellant zone with the medication zone, and

(e) piston means slidably arranged within the channel, so that upon ignition of the propellant gas pressure generated by combustion of the propellant causes displacement of the piston means which then exert pressure on the liquid medication and eject it through the outlet orifice of the nozzle.

### BRIEF DESCRIPTION OF THE DRAWINGS

The subject of the invention will now be described in terms of its preferred embodiments with reference to the accompanying drawings. These embodiments are set forth to aid the understanding of the invention, but are not to be construed as limiting.

FIG. 1 shows a cross-sectional view of a first embodiment of an injection device 1 according to the invention.

FIGS. 2, 3 and 4 show a series of cross-sectional views illustrating the operation of device 11 shown by FIG. 1.

FIGS. 5 and 6 show exploded cross-sectional views of device 11 from different points of view.

FIGS. 7 and 8 show exterior views of device 11 from different points of view.

FIG. 9 shows a cross-sectional view of an injection device having a second embodiment of ignition means.

FIGS. 10, 11 and 12 show a series of cross-sectional views illustrating the operation of the device shown by FIG. 9.

FIGS. 13 and 14 show exploded cross-sectional views of the device shown by FIG. 9 from different points of view.

FIG. 15 shows a cross-sectional view of an injection device having a third embodiment of ignition means.

FIGS. 16, 17 and 18 show a series of cross-sectional views illustrating the operation of the device shown by FIG. 15.

FIGS. 19 and 20 show exploded cross-sectional views of the device shown by FIG. 15 from different points of view.

FIG. 21 shows a cross-sectional view of an injection device having a fourth embodiment of ignition means.

FIGS. 22, 23 and 24 show a series of cross-sectional views illustrating the operation of the device shown by FIG. 21.

FIGS. 25 and 26 show exploded cross-sectional views of the device shown by FIG. 21 from different points of view.

FIGS. 27 and 28 show exterior views of device shown by FIG. 21 from different points of view.

FIG. 29 shows a cross-sectional view of an injection device having a fifth embodiment of ignition means.

FIGS. 30 and 31 show cross-sectional views illustrating the operation of the device shown by FIG. 29.

FIGS. 32 and 33 show exploded cross-sectional views of the device shown by FIG. 29 from different points of view.

FIGS. 34 and 35 show exterior views of device shown by FIG. 29 from different points of view.

FIG. 36 shows a cross-sectional view of an injection device having a sixth embodiment of ignition means.

FIGS. 37, 38 and 39 show a series of cross-sectional views illustrating the operation of the device shown by FIG. 36.

FIGS. 40 and 41 show exploded cross-sectional views of the device shown by FIG. 36 from different points of view.

FIGS. 42 and 43 show exterior views of device shown by FIG. 36 from different points of view.

FIG. 44 shows a cross-sectional view of an injection device having a seventh embodiment of ignition means.

FIGS. 45 and 46 show cross-sectional views illustrating the operation of the device shown by FIG. 44.

FIGS. 47 and 48 show exploded cross-sectional views of the device shown by FIG. 44 from different points of view.

FIGS. 49 and 50 show exterior views of device shown by FIG. 44 from different points of view.

FIG. 51 shows a cross-sectional view of an injection device having an eighth embodiment of ignition means.

FIGS. 52 and 53 show cross-sectional views illustrating the operation of the device shown by FIG. 51.

FIGS. 54 and 55 show exploded cross-sectional views of the device shown by FIG. 51 from different points of view.

FIGS. 56 and 57 show exterior views of device shown by FIG. 51 from different points of view.

FIG. 58 shows a cross-sectional view of an injection device having a ninth embodiment of ignition means.

FIGS. 59, 60 and 61 show cross-sectional views illustrating the operation of the device shown by FIG. 58.

FIGS. 62 and 63 show exploded cross-sectional views of the device shown by FIG. 58 from different points of view.

FIG. 64 shows a cross-sectional view of an injection device having a breakable membrane containing burning propellant.

FIG. 65 shows a cross-sectional view of an injection device having medication partly contained in a rigid shell and nozzle, and expelled by a diaphragm.

FIG. 66 shows a cross-sectional view of an injection device having medication contained in a rigid shell and nozzle, and expelled by a piston.

FIG. 67 shows a cross-sectional view of an injection device having a modular pyrotechnic system.

FIG. 68 shows a cross-sectional view of the modular pyrotechnic system of FIG. 67.

FIG. 69 shows a cross-sectional view of a prior art stab ignition primer.

FIG. 70 shows a cross-sectional view of a stab ignition primer module incorporating a bistable spring and firing pin.

FIG. 71 shows a cross-sectional view of an ignition primer module incorporating a friction firing pin.

### REFERENCE NUMERALS IN DRAWINGS

1 first embodiment injection device

2 two side open stab primer

3 propellant chamber

4

5 firing pin

6 spring

7 release lever

8 breakable crimp joint

9 bore

10 cup seal

11 cup seal clearance hole

12 medication container

13 medication unit

14 flexible wall of medication container 12

15 nozzle

16 fluid channel

17 orifice/jet outlet

18 deformable/elastic barrier

19 break-off protective cap

20 first housing part

21 second housing part

22 seat second housing part 21

23 free volume

24

25

26

27

28 intermediate support member

29

30 screw connection of housing parts

31 first chamber

32 second chamber

33 first zone of first chamber 31

34 second zone of first chamber 31

35

36

37 second housing part

38 hole in second housing part 37

39 seat in second housing part 37

40 impact plunger

41 propellant chamber

42 stab pin

43 support bridge in propellant chamber 41

44 flow passages

45 one side open stab primer

46 bore in propellant chamber 41

47 second embodiment injection device

48 primer shell

49 free volume

50 third embodiment injection device

51 impact plunger

52 tapered section of impact plunger 51

53 hook of impact plunger 51

54 release lever of impact plunger 51

55 cylindrical guide sleeve of rear housing 21

56 release latch

57 conical cavity in release latch 56

58 second housing part

59 hole in second housing part 58

60 fourth embodiment injection device

61 firing pin

62 bistable disk spring

63 actuation screw

64 propellant chamber

65 outer edge of bistable disk spring 62

66 seat in second housing part 58

67 hole in second housing part 68

68 second housing part 58

69

70 fifth embodiment injection device

71 bistable disk spring

72 vent holes in bistable disk spring 71

73 activation push pin

74 rubber seal

75 second housing part

76 outer edge of bistable disk spring 71

77 hole in second housing part 75

78 enlarged head of activation push pin 73

79

80 sixth embodiment injection device

81 firing pin

82 seal bushing

83 propellant chamber

84 clearance holes in seal bushing 82

85 spring retainer washer

86 shoulder of firing pin 81

87 break region of firing pin 81

88 release lever

89 front end of primer 2

90 rear end of primer 2

91 spring

92 connection joint

93 flange of firing pin 81

94 free volume

95 seal joint

96 second housing part

97

98

99

100 seventh embodiment injection device

101 firing pin

102 spring

103 free volume

104 propellant chamber

105 flange of firing pin 101

106 latch ears of firing pin 101

107 twist shaft of firing pin 101

108 shaft seal

109 second housing part

110 hole in second housing part 109

111 support shoulders of propellant chamber 104

112 release grooves of propellant chamber 104

113

114

115

116

117

118

119

120 eighth embodiment injection device

121 firing pin

122 twist shaft

123

124 flange of firing pin 121

125 latch ears of firing pin 121

126 clutch slot of firing pin 121

127 seal flange of twist shaft 122

128 hole in second housing part 130

129 clutch blade of twist shaft 122

130 second housing part

131 spring

132 free volume

133

134

135

136

200 ninth embodiment injection device

201 firing pin

202 propellant chamber

203 ratchet grooves of firing pin 201

204 second housing

205

206 ratchet fingers

207 seal

208

209

210 free volume

211 external impact motion

212 annular volume

213

214

215

220 injection device with breakable membrane

221 breakable membrane

230 injection device with pyrotechnic module

231 nozzle body

232 rigid housing

233 volume enclosed by nozzle body 231 and rigid housing 232

234 first deformable diaphragm

235 cavity of nozzle body 231

236 medication chamber

237 medication

238 second deformable diaphragm

239 chamber enclosed by second deformable diaphragm 238 and rigid housing 232

240 propellant and ignition means

241 channel of nozzle body 231

242 orifice of nozzle body 231

243 vents

244 space between first deformable diaphragm 234 and second deformable diaphragm 238

245 first deformable diaphragm

246 shell formed by nozzle body 231 and rigid housing 232

250 injection device with piston

251 rigid medication chamber

252 medication zone of rigid medication chamber 251

253 liquid medication

254 nozzle end of rigid medication chamber 251

255 outlet orifice

256 cylindrical bore of rigid medication chamber 251

257 rigid housing

258 cylindrical bore of rigid housing 257

259 drive piston

260 propellant zone formed by rigid housing 234 and second deformable diaphragm 238

261 propellant and ignition means

262 medication piston

263

264

270 injection device with pyrotechnic module

271 pyrotechnic module

272 connection

300 prior art two side open stab primer

301 metallic shell

302 impact and friction sensitive primer

303 secondary ignition material layer

304 propellant layer

305 paper or foil layer

306 schematic needle

307 first open end of metallic shell 301

308 second open end of metallic shell 301

320 modular stab igniter

321 bistable spring

322 firing pin

323 spacer ring

324 external force

325

340 modular friction igniter

341 friction firing pin

342 support disk

343 impact and friction sensitive primer layer

344 secondary ignition material layer

345 propellant layer

346 serrations of friction firing pin 341

347

### DETAILED DESCRIPTION OF PREFERRED EMBODIMENTS

Various embodiments of a device according to the invention for delivering a needleless hypodermic injection of a liquid medication contained therein are described hereinafter with reference to the accompanying figures. Each of these embodiments comprises pyrotechnical means for generating within the device a pressure necessary for injecting the medication. For this purpose, the embodiments described hereinafter comprise non-electric ignition means utilizing stab primers for igniting a propellant contained in the device.

The terms “free volume”, “total volume” and “surface area” used in the following description of preferred embodiments have the following meanings within the context of the instant invention:

The components of an injection device according to the invention are contained in a shell which is configured and dimensioned to withstand the injection pressure generated within the device by combustion of a propellant. Such a cell is called the pressurized shell of the injection device.

The term “free volume” stands for the initial volume within the pressurized shell that is neither occupied by solid material such as metal, polycarbonate, or polyethylene, nor by fluid. Therefore, it is that part of the volume within the pressurized shell into which the combustion gases can extend immediately after ignition. The “total volume” at the end of the injection is the initial “free volume” plus the volume of the liquid ejected from the cartridge. The “surface area” is the area of solid materials contacted by the combustion gases.

The above-defined “free volume”, “total volume” and “surface area” affect the performance of the injection device in the following ways:

The “free volume” and the mass of propellant determine the maximum value reached by the initial injection pressure after ignition. If the “free volume” is increased, an increased propellant mass is required to reach a given initial injection pressure.

The pressure drops from the initial injection pressure to the end of injection pressure because of the mechanical work done as the gas expands from the “free volume” to the “total volume”. The pressure also drops because of heat transfer to the “surface area”.

The “free volume” and the propellant quantity are adjusted to provide the required initial injection pressure and end of injection pressure. As “surface area” and resulting heat losses increase, the “free volume” and propellant mass must be increased to compensate. Minimization of “surface area” therefore leads to minimum “free volume”and propellant mass. Reduction of propellant mass reduces the size and cost of the pressure containment structure required for safe operation, and makes single use disposable injectors economically attractive.

### Embodiment 1

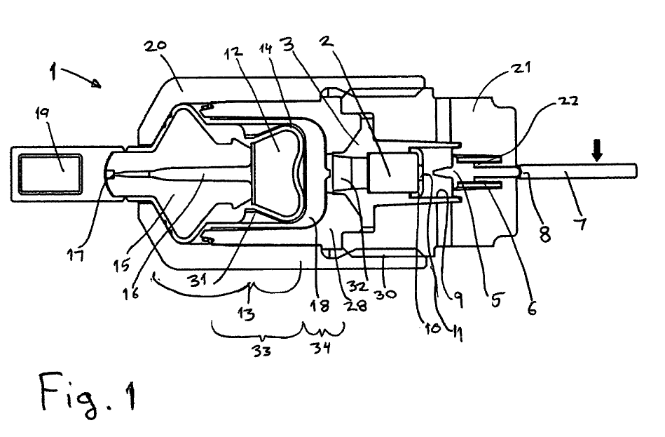

FIG. 1 shows a first embodiment of a device 1 according to the invention. Device 1 is a cartridge characterized in that the ignition means contained therein comprise a two-side-open primer 2 and a firing pin 5 for striking primer 2. Primer 2 is positioned within a propellant chamber 3 that supports the outside diameter of primer 2 and locates it axially. Firing pin 5 is urged towards primer 2 by compressed spring 6, and held in this cocked position by release lever 7 attached to firing pin 5 by the breakable crimp joint 8. Propellant chamber 3 includes a cylindrical bore 9 that holds a resilient cup seal 10 adjacent to the primer 2. Cup seal 10 has a central hole 12 large enough in diameter that the firing pin 5 may pass through and strike the primer 2. Preferably, primer 2 contains the full quantity of propellant required for the injection. Optionally, additional propellant may be placed in chamber 32.

Primer 2 is preferably a “green igniter” of the type that avoids pollution problems and toxicity issues due to toxic heavy metals contained in “non-green” igniters.

An important advantage of the first embodiment of the ignition means is that it can be manufactured at low cost for two principal reasons. First, manufacture of stab primers 2 uses technology and production capacity established to serve existing military and civilian markets. It may be purchased as a sealed, assembled module that includes all the required propellant. Second, the mechanical ignition energy requirement of less than 10 millijoules results in small, low-cost firing pins 5, springs 6 and release levers 7. The spring illustrated in FIG. 1 (Associated Spring part number CO 180-016-0440), for example, has an outside diameter of 4.57 millimeters, a free length of 9.65 millimeters, and wire diameter of 0.4 millimeters. It stores 11.9 millijoules of mechanical energy.

Ignition of primer 2 preferably produces the required hot gas on its own. Optionally, it lights additional propellant contained in the chamber 32 to generate additional hot gas.

Additional propellant optionally contained within device 1 is, for example, a fine grain nitrocellulose based composition or another nitrocellulose based composition, or another propellant composition having similar properties or a mixture of propellant compositions.

Two end open stab primer 2 is preferably closed at each end by moisture seals that open at low pressure and exclude contact of the primer material with water vapor prior to use of the device.

When the pressure reaches a predetermined level, the burst diaphragm opens and the pressure generated by combustion of the primer and propellant causes ejection of the medication out of the device for performing an injection.

Injection device 1 described above can have different structures, and the illustrated structure is only an example.

FIG. 1 shows a cross-sectional view of a structure of device 1 designed as a single use device, that is a device which is used only once and discarded after use.

Injection device 1 shown by FIG. 1 comprises a housing consisting of a first housing part 20, e.g. an aluminum shell, and a second housing part 21, made of, for example, a polycarbonate. Housing parts 20 and 21 have threads which match with each other and are thus be connected with each other by a screw connection 30.

In a preferred embodiment, the housing of device 1 is so configured and dimensioned that as a whole it is adapted to withstand an internal pressure which is higher than the normal injection pressure without yielding.

In a preferred embodiment, both parts 20 and 21 of the housing of device 11 are made of suitable plastic materials, e.g., from commercially available polyesters or polycarbonates taking in particular into account the mechanical properties the housing should have.

The interior of the housing of device 11 comprises a first chamber 31 and a second chamber 32, which are defined for instance by respective cavities of a support member 28.

A medication unit 13 comprising nozzle 15 and medication container 12 formed by deformable wall 14 is arranged within first chamber 31. A volume of liquid to be injected is stored in medication unit 13. In preferred embodiments, the amount of this volume is in a range from about 50 to about 1000 microliters. Specific examples of this amount are, for example, 200 or 500 microliters.

Medication unit 13 is a sealed medication module which comprises a nozzle body 15 and a flexible container wall 14 that hermetically encloses a portion of the nozzle and forms a reservoir 12 for a liquid medication stored in sealed medication unit 13. Wall 14 is deformable and collapsible.

Medication unit 13 thus comprises a first region and a second region that are in liquid communication with each other. The first region is deformable and comprises the reservoir enclosed by flexible wall 14. The second region of medication unit 13 comprises nozzle 15, which has a fluid channel 16 that ends in an orifice 17, which serves as a liquid jet outlet through which liquid to be injected is ejected when an injection is performed with injector module 11. Medication unit 13 is made of one or more suitable construction materials, e.g., polyethylene and polypropylene, which are suitable for storing medications including sensitive protein drugs.

A part of container wall 14 forms a break-off protective cap 19 that covers a jet orifice 17 of nozzle body 15. Cap 19 is removed by the user just prior to use of injector module 11.

First chamber 31 comprises two zones, a first zone 33 which contains medication unit 13 and a second zone 34 which is located between medication unit and second chamber 32. First chamber 31 is in communication with second chamber 32 so that upon ignition of stab primer 2 located within second chamber 32, gas thereby generated expands into second zone 34 of first chamber 31, exerts pressure on and deforms deformable wall 14 of the first region of medication unit 13 and thereby causes ejection of the liquid medication through channel 16 and orifice 17.

In a preferred embodiment, a deformable/elastic barrier 18 divides the first zone 33 from the second zone 34. The elastic barrier is made of materials like, for example, silicon rubber, and can be reinforced with materials like, for example, woven aramid fibers.

In a preferred embodiment the free volume comprised between medication unit 13 and the wall of support member 28 is much smaller than the volume available for containing propellant within the device.

FIGS. 2 through 4 illustrate the operating sequence of needleless injection device 1.

FIG. 2 shows needleless injection device 1 prior to the injection. The removable cap 19 is broken off to uncover the sterile injection nozzle 17, and the nozzle is pressed against the patient's skin at the injection site.

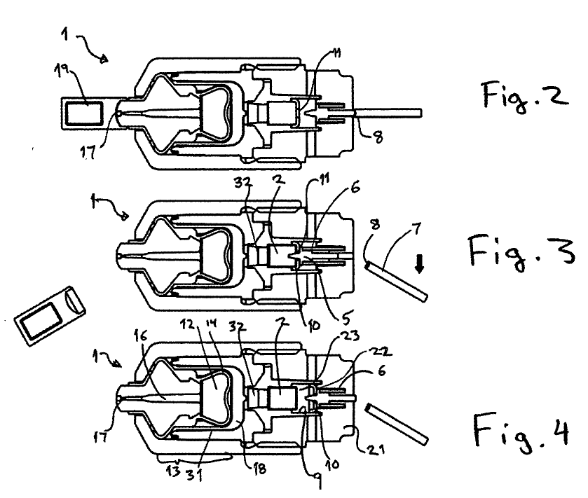

FIG. 3 shows needleless injection device 1 at the instant of ignition. As a result of pushing the release lever 7 to one side, the breakable crimp joint 8 separates and releases firing pin 5. The compressed spring 6 accelerates firing pin 5 so that its tip passes through hole 11 in cup seal 10 and strikes and ignites primer 2. The hot products of combustion of primer 2 ignite any optional propellant contained in chamber 32. The release lever 7 may be pushed directly by the user or indirectly through a mechanism that is part of an application device (not shown).

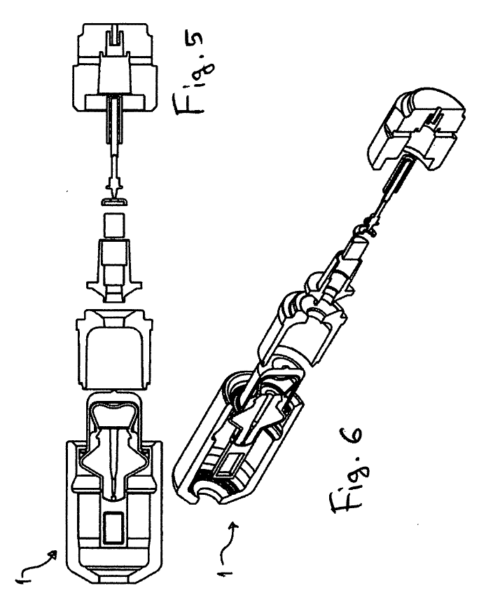

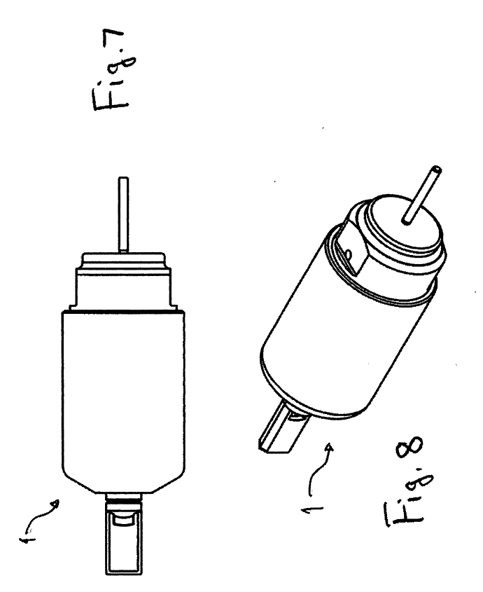

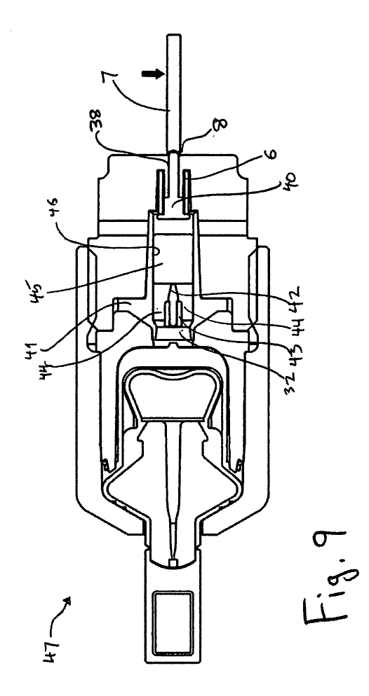

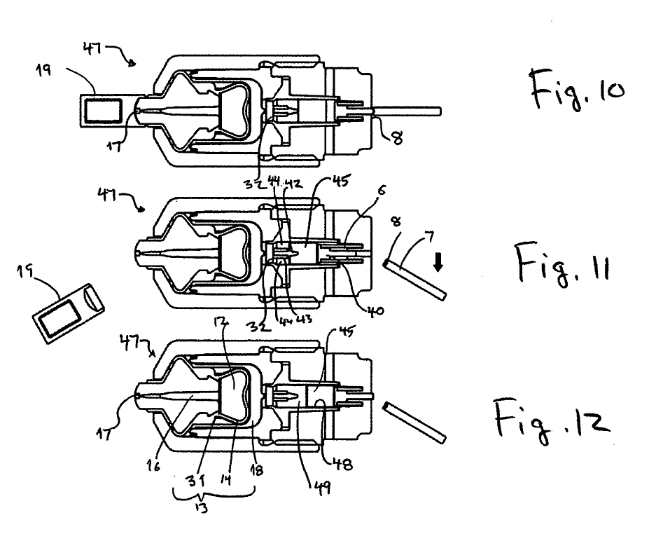

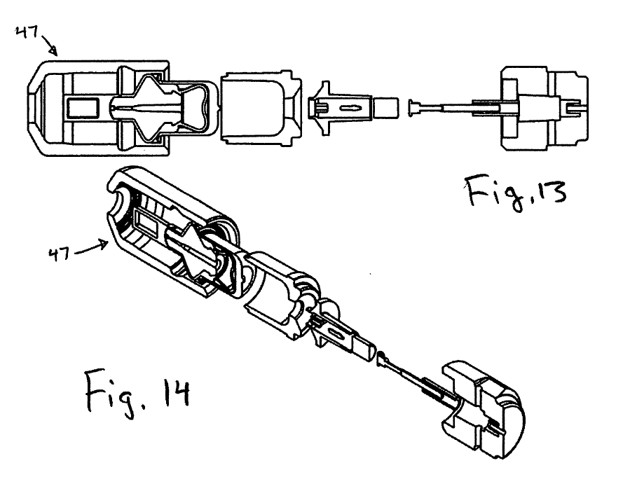

FIG. 4 shows needleless injection device 1 an instant following ignition. Hot gas flows from the front opening of the two opening stab primer 2 into chamber 31 through chamber 32. Chamber 32 may contain additional propellant of the same or a different type that burns and adds to the hot gas. The hot gas in chamber 31 applies pressure to the liquid medication unit 12 through the flexible wall 14 of the medication unit 13 and through the deformable elastic barrier 18. This pressure forces the liquid medication unit 12 through fluid channel 16 and out the injection nozzle 17, which forms the skin-penetrating jet. The pressure rises to a maximum initially, and drops to lower values as the injection proceeds to completion. At the same time, hot gas flows from the rear opening of the two opening stab primer 2 and forces back the cup seal 10 and firing pin 5 through the bore 9 until the firing pin contacts and seals against seat 22. The combined effects of the cup seal 10 and the firing pin 5 are to contain the hot gas in free volume 23, and prevent unwanted flow into the volume surrounding the spring 6 and the annular space between the firing pin 5 and the second housing part 21. Such unwanted flow would result in loss of efficiency through an increase in dead volume, heat losses to solid surfaces, and external leakage.

FIGS. 5 and 6 show exploded cross-sectional views of components of device 1 shown by FIGS. 1 through 4 from different points of view. FIGS. 7 and 8 show external views of device 1 shown by FIGS. 1 through 6 from different points of view.

FIGS. 9 to 71 show seven additional embodiments of stab primer ignition means suitable for use as part of an injection device having a structure similar to the structure of the above described injection device 1 in FIG. 1. The descriptions of the additional embodiments include only the new elements, and do not repeat the description of the complete device.

In particular, an advantage of the above-described embodiment 1 is that there is no contact between the hot gas generated in the propellant chamber and the spring used for driving the firing pin. Therefore, there is no loss of energy that would be caused by such a contact. Another advantage of embodiment 1 is that the structure of the device ensures a good sealing of the propellant chamber and this sealing is assisted by the pressure generated within the propellant chamber. Therefore, there is no loss of pressure built up in the propellant chamber that would be due to lack of a good sealing thereof. Thus, the whole pressure generated within the propellant chamber is available for being effectively used as injection pressure.

### Embodiment 2

FIG. 9 shows a sectional view of an injection device 47 having a similar structure as injection device 1 shown in FIG. 1, but wherein one side open stab primer 45 is utilized.

One side, open stab primer 45 is slidably mounted in bore 46 of the propellant chamber 41, and is oriented such that the open side faces the sharp point of stab pin 42. Stab pin 42 is concentric with primer 45, and is attached to support bridge 43 that crosses chamber 32 of propellant chamber 41. Flow passages 44 on each side of support bridge 43 connect the open side of primer 45 to chamber 32 and chamber 31. Impact plunger 40 is positioned adjacent to the closed side of the primer 45, and slideably held in hole 38 in second housing part 37. Impact plunger 40 is urged towards primer 45 by compressed spring 6, and held in this cocked position by release lever 7 attached to impact plunger 40 by the breakable crimp joint 8. Preferably, primer 45 contains the full quantity of propellant required for the injection. Optionally, additional propellant may be placed in chamber 32.

Primer 45 is preferably a “green igniter” of the type that avoids pollution problems and toxicity issues due to toxic heavy metals contained in “non-green” igniters.

FIGS. 10 through 12 illustrate the operating sequence of needleless injection device 47.

FIG. 10 shows needleless injection device 47 prior to the injection. The removable cap 19 is broken off to uncover the sterile injection nozzle 17, and the nozzle is pressed against the patient's skin at the injection site.

FIG. 11 shows needleless injection device 47 at the instant of ignition. As a result of pushing the release lever 7 to one side, the breakable crimp joint 8 separates and releases impact plunger 40. The compressed spring 6 accelerates impact plunger 40 so that it contacts and accelerates primer 45 toward stab pin 42, such that stab pin 42 penetrates and ignites primer 45. The hot products of combustion of primer 45 pass around support bridge 43 through flow passages 44, and ignite any optional propellant contained in chamber 32. The release lever 7 may be pushed directly by the user or indirectly through a mechanism that is part of an application device (not shown).

FIG. 11 shows needleless injection device 47 an instant following ignition. Hot gas flows from the front opening of the one opening stab primer 45 into chamber 31 through chamber 32 and flow passages 44. Chamber 32 may contain additional propellant of the same or a different type that burns and adds to the hot gas. The hot gas in chamber 31 applies pressure to the liquid medication unit 12 through the flexible wall 14 of the medication unit 13 and through the deformable elastic barrier 18. This pressure forces the liquid medication unit 12 through fluid channel 16 and out the injection nozzle 17 which forms the skin-penetrating jet. The pressure rises to a maximum initially, and drops to lower values as the injection proceeds to completion. At the same time, hot gas pressure on the empty primer shell 48 of the one opening stab primer 45 forces back the primer shell 48 and impact plunger 40 through the bore 46 until the impact plunger contacts seat 39. Gas pressure expands the primer shell 48 so that it seals against bore 46. The combined effects of the primer shell 48 and the impact plunger 40 are to contain the hot gas in free volume 49, and prevent unwanted flow into the volume surrounding the spring 6 and the annular space between the impact plunger 40 and the second housing part 37. Such unwanted flow would result in loss of efficiency through an increase in dead volume, heat losses to solid surfaces, and external leakage.

FIGS. 13 and 14 show exploded cross-sectional views of components of device 47 shown by FIGS. 9 through 12 from different points of view.

An advantage of the above-described embodiment 2 over embodiment 1 is that the shell of the stab primer effectively seals the propellant chamber and no additional sealing means is required for sealing this chamber. Thus, embodiment 2 and the manufacturing process for making it are cheaper. Moreover, due to the good sealing of the propellant chamber ensured by the shell of the stab primer, there is no loss of pressure built up in the propellant chamber that would be due to lack of a good sealing thereof. Thus, the whole pressure generated within the propellant chamber is available for being effectively used as injection pressure. As in the case of embodiment 1, there is also no contact between the hot gas generated in the propellant chamber and the spring used for driving the firing pin. Therefore, there is no loss of energy that would be caused by such a contact.

### Embodiment 3

FIG. 15 shows a sectional view of an injection device 50 having a similar structure and ignition means as injection device 47 shown in FIG. 9, but wherein a different impact plunger release mechanism is employed.

Impact plunger 40 in FIG. 9 is replaced by impact plunger 51 having a tapered section 52, a hook 53, and a release lever 54. Hook 53 engages the second housing part 58 when impact plunger 51 is pulled back to compress spring 6 and pushed to one side in hole 59. This holds impact plunger 51 in the cocked position, and spring 6 tilts to accommodate the resulting angularity. Cylindrical guide sleeve 55 is preferably formed as part of second housing part 58, and encircles release lever 54. Release latch 56 is a cylindrical rod with a conical cavity 57 in one end. Release latch 56 is a sliding fit within guide sleeve 55. Guide sleeve 55 also protects release lever 54 from accidental contact and release during production handling. Release latch 56 is preferably part of a separate application device (not shown), and not part of injection device 50.

FIG. 16 shows needleless injection device 50 prior to the injection. The removable cap 19 is broken off to uncover the sterile injection nozzle 17, and the nozzle is pressed against the patient's skin at the injection site.

FIG. 17 shows needleless injection device 50 at the instant of ignition. As a result of pushing release latch 56 into guide sleeve 55, the conical cavity 57 contacts the release lever 54 and forces it to center. This action disengages hook 53 from second housing part 58, and releases impact plunger 51. As described for injection device 47 shown in FIG. 9, the ignition process proceeds.

FIG. 18 shows needleless injection device 50 an instant following ignition. As described for injection device 47 shown in FIG. 9, the propellant combustion and injection process proceeds. At the same time, hot gas pressure on the empty primer shell 48 of the one opening stab primer 45 forces back the primer shell 48 and impact plunger 51 through the bore 46 until the impact plunger contacts seat 66. Gas pressure expands the primer shell 48 so that it seals against bore 60. The combined effects of the primer shell 48 and the impact plunger 51 are to contain the hot gas in free volume 49 and prevent unwanted flow into the volume surrounding the spring 6 and the annular space between the impact plunger 51 and the second housing part 58. Such unwanted flow would result in loss of efficiency through an increase in dead volume, heat losses to solid surfaces, and external leakage.

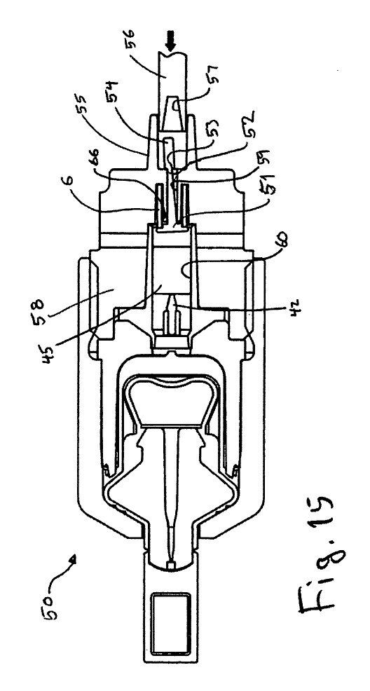

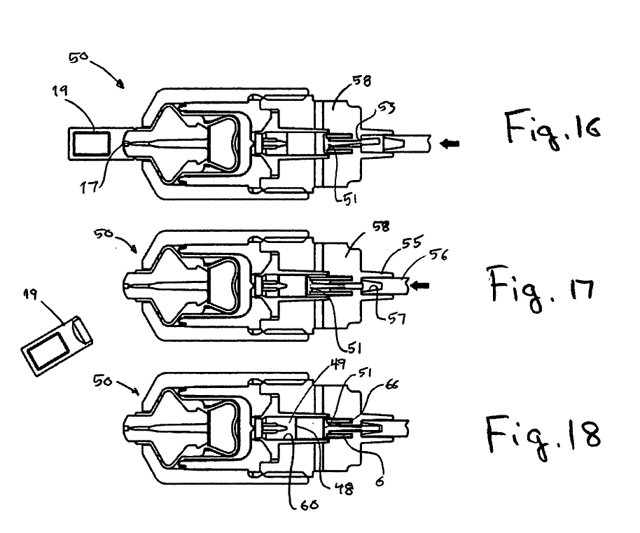

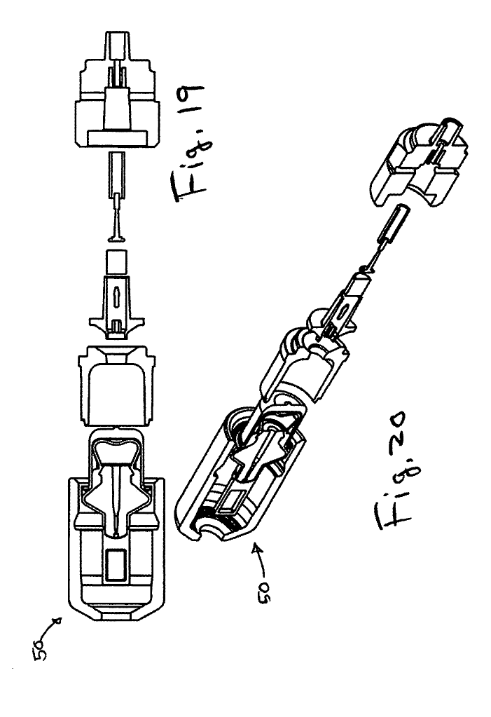

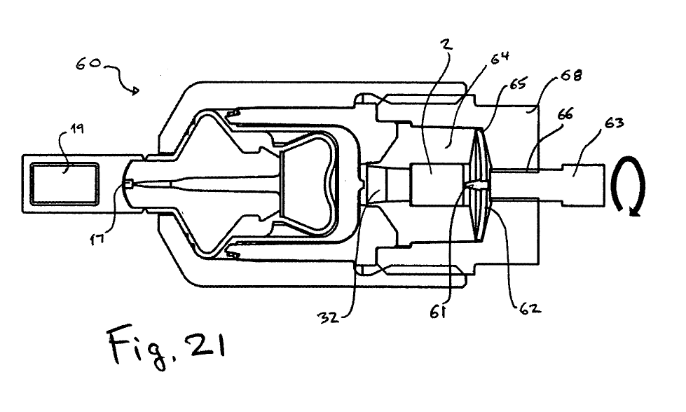

FIGS. 19 and 20 show exploded cross-sectional views of components of device 50 shown by FIGS. 15 through 18 from different points of view.

An advantage of the above-described embodiment 3 over embodiment 2 is that the release motion, i.e., the motion of release latch 56, is in jet direction, and this makes use of the injection device easier than when the release motion is in other directions.

### Embodiment 4

FIG. 21 shows a sectional view of an injection device 60 having a similar structure as injection device 1 shown in FIG. 1, but wherein firing pin 5 and spring 6 in device 1 are replaced by firing pin 61 and bistable disk spring 62.

The ignition means comprises two side open primer 2 positioned within a propellant chamber 64 that supports the outside diameter of primer 2 and locates it axially. Firing pin 61 is attached to the center of bistable disk spring 62, such that the sharp end faces the center of primer 2. Bistable disk spring 62 is supported at its outer edge 65 by an annular pivot formed by opposing surfaces of propellant chamber 64 and second housing part 68. It is shown in a first stable position in which it is concave away from primer 2, and firing pin 61 does not contact primer 2. Actuation screw 63 is threaded into hole 66 in second housing part 68, and is arranged such that it may be turned to push the center of bistable disk spring 62 and firing pin 61 toward primer 2. Preferably, primer 2 contains the full quantity of propellant required for the injection. Optionally, additional propellant may be placed in chamber 32.

FIGS. 22 through 24 illustrate the operating sequence of needleless injection device 60.

FIG. 22 shows needleless injection device 60 prior to the injection. The removable cap 19 is broken off to uncover the sterile injection nozzle 17, and the nozzle is pressed against the patient's skin at the injection site.

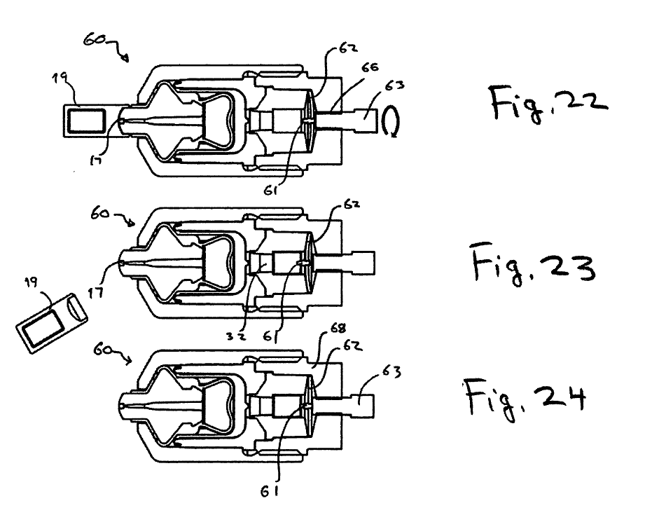

FIG. 23 shows needleless injection device 60 at the instant of ignition. As a result of turning the actuation screw 63, the bistable disk spring 62 and firing pin 61 are pushed toward primer 2. At a transition point, the bistable disk spring 62 snaps to a second stable position causing the firing pin 61 to penetrate and ignite primer 2. The hot products of combustion of primer 2 ignite any optional propellant contained in chamber 32. The actuation screw 63 may be turned directly by the user or indirectly through a mechanism that is part of an application device (not shown).

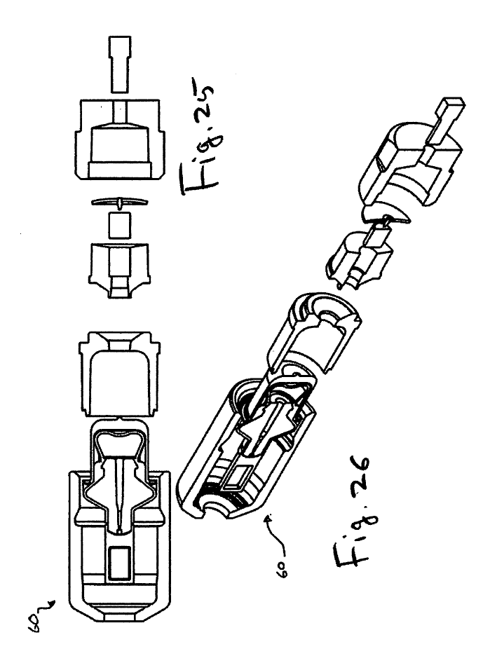

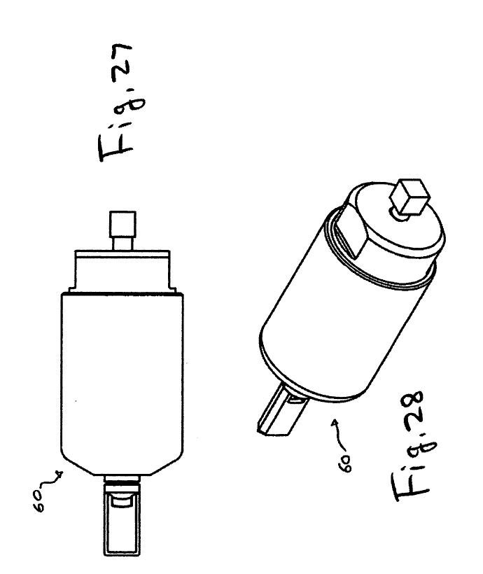

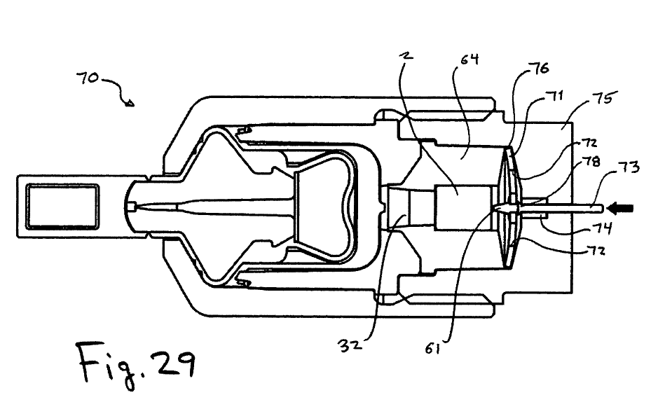

FIG. 24 shows needleless injection device 60 an instant following ignition. As described for injection device 1 shown in FIG. 1, the propellant combustion and injection process proceeds. At the same time, hot gas flows from the rear opening of the two opening stab primer 2 and forces back the bistable disk spring 62 and firing pin 61 against the actuation screw 63 at the center and the second housing part 68 at the outer edge. This forms a barrier and seal that blocks hot gas flow to the vicinity of the actuation screw 63.

FIGS. 25 and 26 show exploded cross-sectional views of components of device 60 shown by FIGS. 21 through 24 from different points of view. FIGS. 27 and 28 show external views of device 60 shown by FIGS. 21 through 26 from different points of view.

The above-described embodiment 4 offers the following advantages:

safety of operation is improved by the fact that 53 blocks the rear of device 60 and that the device does not have any fast moving parts outside the device,

the structure of the device is very compact and this makes possible to obtain a high gas pressure,

after ignition of the propellant, there is a very limited increase of the volume available for the gas generated within the device,

only a relatively small surface is in contact with combustion gases, i.e. heat loss is very low and thereby loss of injection pressure generated by combustion of the propellant is also very low,

disk spring 62 can be a non-metallic one, e.g. a material with a low elasticity modulus, and

since spring 62 serves as seal and provides a good sealing of the propellant chamber, no other sealing means is required.

### Embodiment 5

FIG. 29 shows a sectional view of an injection device 70 having a similar structure as injection device 60 shown in FIG. 21, but wherein a different spring and release mechanism is employed.

Bistable disk spring 62 in device 60 that provides a gas barrier function is replaced in injection device 70 by bistable disk spring 71, that includes vent holes 72 that equalizes pressure on both sides of the disk spring. Actuation screw 63 in device 60 is replaced by small diameter actuation push pin 73 with enlarged diameter head 78 on one end. Rubber seal 74 is added between push pin 73 and second housing part 75 to block gas flow out of device 70. The ignition means comprises two side open primer 2 positioned within a propellant chamber 64 that supports the outside diameter of primer 2 and locates it axially. Firing pin 61 is attached to the center of bistable disk spring 71, such that the sharp end faces the center of primer 2. Bistable disk spring 71 is supported at its outer edge 76 by an annular pivot formed by opposing surfaces of propellant chamber 64 and rear housing 75. It is shown in a first stable position in which it is concave away from primer 2, and firing pin 61 does not contact primer 2. Actuation pin 73 slides into hole 77 in rear housing 75, and is arranged such that it transfers an externally applied force to push the center of bistable disk spring 71 and firing pin 61 toward primer 2. Enlarged diameter head 78 is on the end of the pin inside rear housing 75.

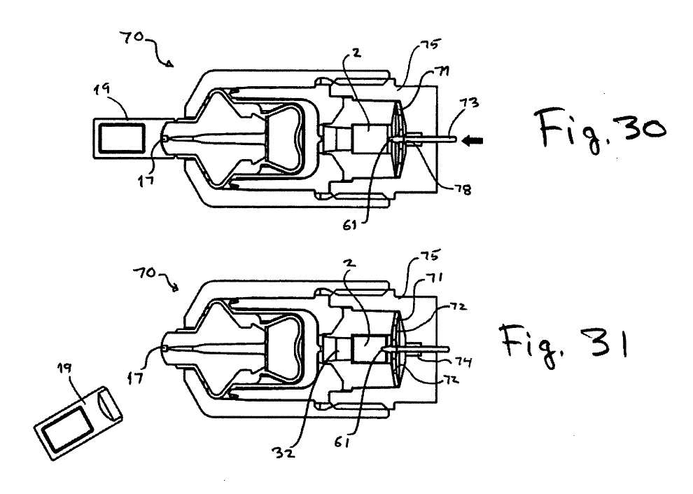

FIGS. 30 and 31 illustrate the operating sequence of needleless injection device 70.

FIG. 30 shows needleless injection device 70 prior to the injection. The removable cap 19 is broken off to uncover the sterile injection nozzle 17, and the nozzle is pressed against the patient's skin at the injection site.

FIG. 31 shows needleless injection device 70 during and after the instant of ignition. As a result of pushing the actuation push pin 73, the bistable disk spring 71 and firing pin 61 are pushed toward primer 2. At a transition point, the bistable disk spring 71 snaps to a second stable position causing the firing pin 61 to penetrate and ignite primer 2. The hot products of combustion of primer 2 ignite any optional propellant contained in chamber 32. Actuation push pin 71 may be pushed directly by the user or indirectly through a mechanism that is part of an application device (not shown). As described for injection device 60 shown in FIG. 21, the propellant combustion and injection process proceeds. At the same time, hot gas flows from the rear opening of two opening stab primer 2 and through vent holes 72 in bistable disk spring 71. This gas pressure acts on actuation push pin 73, and tends to push it out of the hole 77 in rear housing 75. Because of the small diameter of actuation push pin 73, this force is small and is resisted by the external force applied by the user or the actuation device. Head 78 on actuation push pin 73 prevents its ejection in the event that the external force is lower than the pressure force. Rubber seal 74 prevents gas flow through the annular clearance between actuation push pin 73 and rear housing 75.

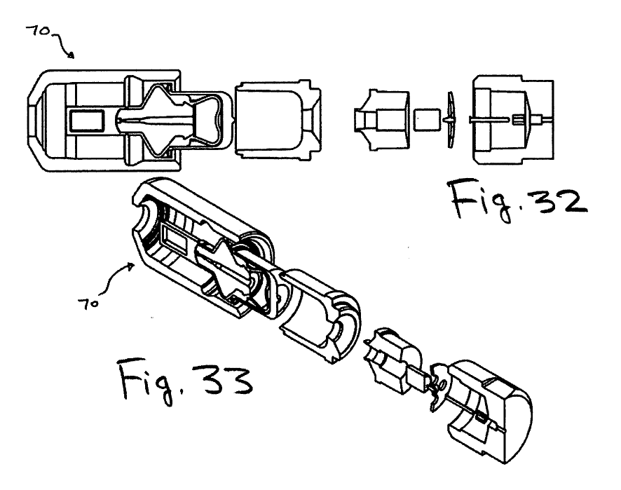

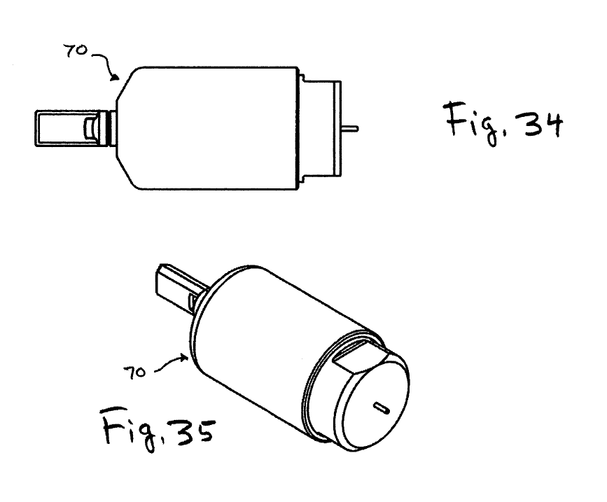

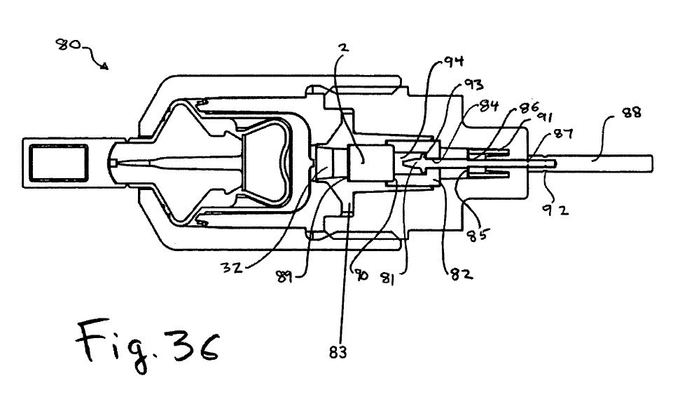

FIGS. 32 and 33 show exploded cross-sectional views of components of device 70 shown by FIGS. 29 through 31 from different points of view. FIGS. 34 and 35 show external views of device 70 shown by FIGS. 29 through 33 from different points of view.

The above described embodiment 5 offers the following advantages:

the actuation motion is in jet direction,

actuation of the ignition means is effected by a linear push motion and there is a natural release point determined by the properties of disk spring 71,

the structure of the device is very compact and this makes possible to obtain a high gas pressure,

after ignition of the propellant there is no increase of the volume available for the gas generated within the device, and

disk spring 71 can be a non-metallic one, e.g. a material with a low elasticity modulus.

### Embodiment 6

FIG. 36 shows a sectional view of an injection device 80 having a similar structure as injection device 1 shown in FIG. 1, but wherein firing pin 5 and cup seal 10 in device 1 are replaced by firing pin 81 and seal bushing 82, with the objective of minimizing the free volume 94 and surface area associated with the firing pin mechanism.

The ignition means comprises two side open primer 2 positioned within a propellant chamber 83 that supports the outside diameter of primer 2 and locates it axially at the front end 89. Seal bushing 82 contains and locates primer 2 at the rear end 90, and has clearance hole 84. Firing pin 81 includes shoulder 86, flange 93 and break region 87. Break region 87 consists, for example, of a groove that locally weakens firing pin 81. Firing pin 81 is urged towards primer 2 by compressed spring 91 that bears against spring retainer washer 85 that in turn bears on shoulder 86. It is held in this cocked position by release lever 88 attached to firing pin 81 by connection joint 92. Firing pin 81 passes through clearance hole 84 in seal bushing 82. Preferably, primer 2 contains the full quantity of propellant required for the injection. Optionally, additional propellant may be placed in chamber 32.

FIGS. 37 through 39 illustrate the operating sequence of needleless injection device 80.

FIG. 37 shows needleless injection device 80 prior to the injection. The removable cap 19 is broken off to uncover the sterile injection nozzle 17, and the nozzle is pressed against the patient's skin at the injection site.

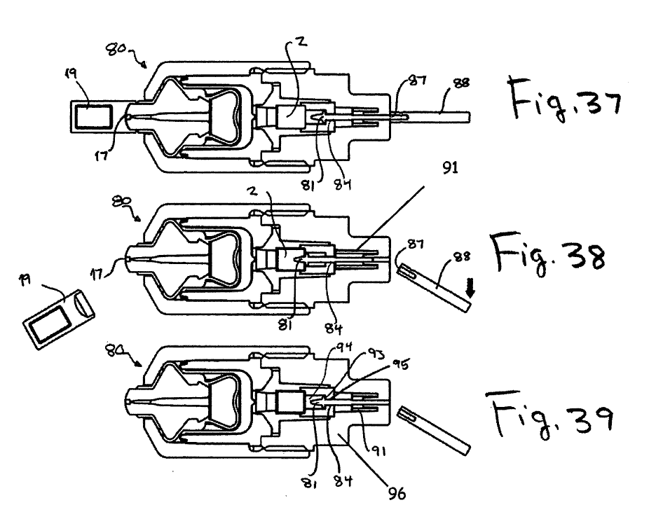

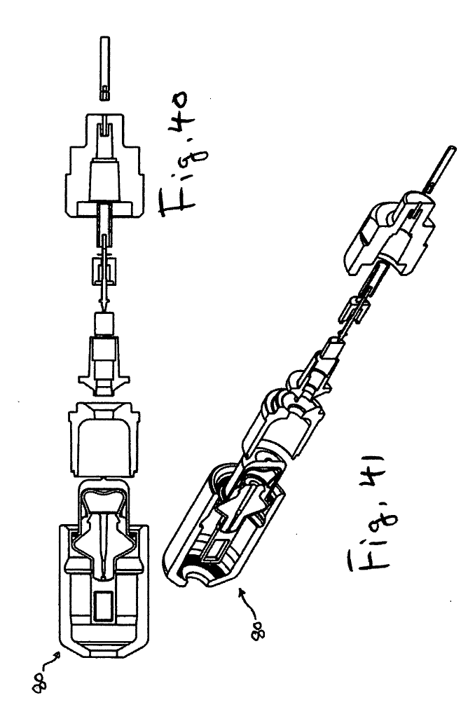

FIG. 38 shows needleless injection device 80 at the instant of ignition. As a result of pushing release lever 88 to one side, break region 87 in firing pin 81 separates and releases firing pin 81. The compressed spring 91 accelerates firing pin 81 so that it slides though clearance hole 84 in seal bushing 82 and strikes and ignites primer 2. The hot products of combustion of primer 2 ignite any optional propellant contained in chamber 32. The release lever 88 may be pushed directly by the user or indirectly through a mechanism that is part of an application device (not shown).

FIG. 39 shows needleless injection device 80 an instant following ignition. As described for injection device 1 shown in FIG. 1, the propellant combustion and injection process proceeds. At the same time, hot gas flows from the rear opening of two opening stab primer 2 and forces back firing pin 81 through clearance hole 84 until flange 93 contacts seal bushing 82 and forms seal joint 95. The effect is to contain the hot gas in free volume 94, and prevent unwanted flow into the volume surrounding spring 91 and the annular space between the firing pin 81 and the second housing part 96. Such unwanted flow would result in loss of efficiency through an increase in dead volume, heat losses to solid surfaces, and external leakage. Embodiment 6 is notable in that free volume 94 may be very small.

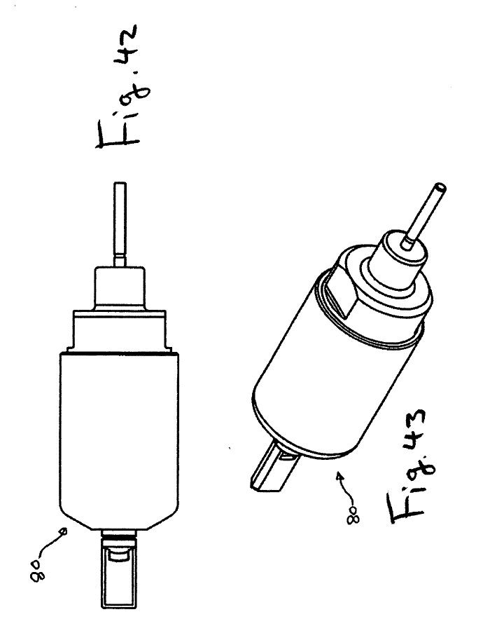

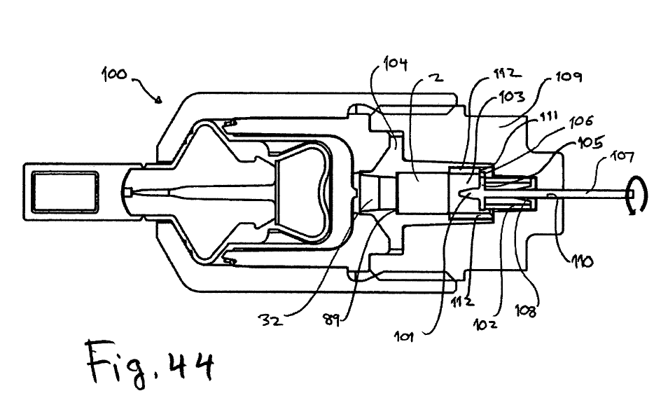

FIGS. 40 and 41 show exploded cross-sectional views of components of device 80 shown by FIGS. 36 through 39 from different points of view. FIGS. 42 and 43 show external views of device 80 shown by FIGS. 36 through 41 from different points of view.

The above described embodiment 6 offers the following advantages:

the volume available for gas expansion behind the igniter is minimized,

only a relatively small surface is in contact with combustion gases, i.e., heat loss is very low and thereby loss of injection pressure generated by combustion of the propellant is also very low, and

the structure of the device ensures a good sealing of the propellant chamber and this sealing is assisted by the pressure generated within the propellant chamber.

### Embodiment 7

FIG. 44 shows a sectional view of an injection device 100 having a similar structure as injection device 1 shown in FIG. 1, but wherein firing pin 101 and spring 102 and the associated release mechanism are contained within free volume 103 that is filled by hot gas

The ignition means comprises two side open primer 2 positioned within a propellant chamber 104 that supports the outside diameter of primer 2 and locates it axially at the front end 89. Propellant chamber 104 also incorporates support shoulders 111 and release grooves 112. Firing pin 101 includes flange 105 with latch ears 106, and a twist shaft 107 that extends through shaft seal 108 and hole 110 in second housing part 109. Twist shaft 107 provides a means of rotating firing pin 101 by a torque applied from outside injection device 100. Compressed spring 102 holds shaft seal 108 in contact with second housing part 109. Firing pin 101 is urged towards primer 2 by compressed spring 102 that bears against flange 105. In a first rotational position, firing pin 101 is held in this cocked position by the engagement of latch ears 106 with support shoulders 111. In a second rotational position, latch ears 106 disengage from support shoulders 111 and align with release grooves 112, freeing firing pin 101 to move axially and make contact with primer 2. Preferably, primer 2 contains the full quantity of propellant required for the injection. Optionally, additional propellant may be placed in chamber 32.

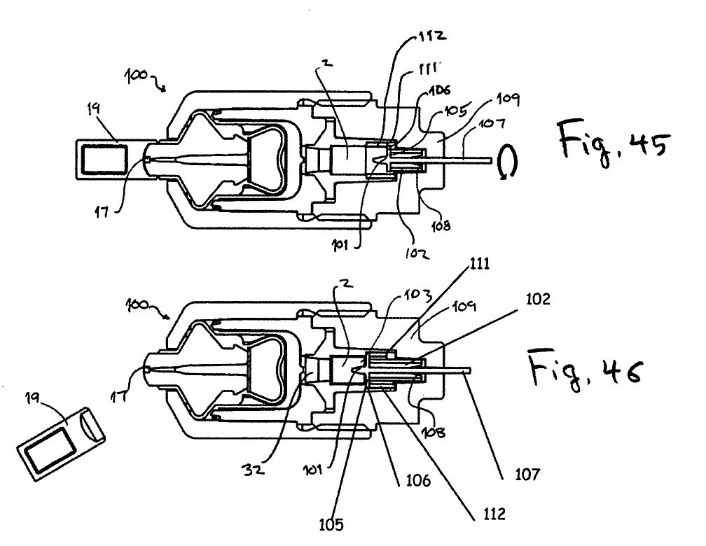

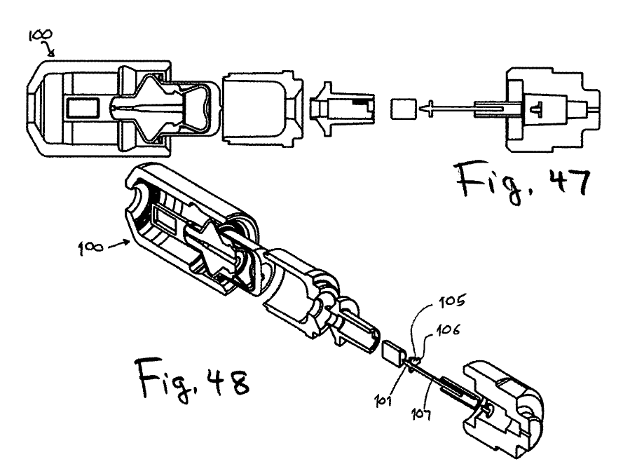

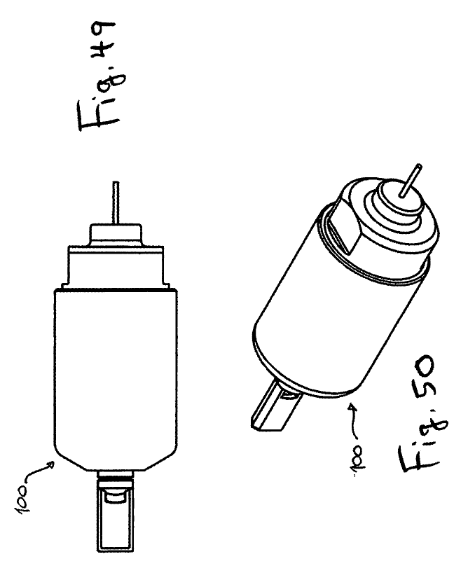

FIGS. 45 and 46 illustrate the operating sequence of needleless injection device 100.

FIG. 45 shows needleless injection device 100 prior to the injection. The removable cap 19 is broken off to uncover the sterile injection nozzle 17, and the nozzle is pressed against the patient's skin at the injection site.

FIG. 46 shows needleless injection device 100 during and after the instant of ignition. As a result of turning twist shaft 107, firing pin 101 shifts from a first rotational position in which it is held in a cocked position by the engagement of latch ears 106 with support shoulders 111, and reaches a second rotational position wherein latch ears 106 disengage from support shoulders 111 and align with release grooves 112. Compressed spring 102 is then free to accelerate firing pin 101 so that it strikes and ignites primer 2. The hot products of combustion of primer 2 ignite any optional propellant contained in chamber 32. As described for injection device 60 shown in FIG. 21, the propellant combustion and injection process proceeds. At the same time, hot gas flows from the rear opening 90 of two opening stab primer 2 and into free volume 103. This gas pressure acts on twist shaft 107, and tends to push it out through hole 110 in second housing part 109. Because of the small diameter of twist shaft 107, this force is small and is resisted by the residual force of spring 102 and external force applied by the user or the actuation device. Flange 105 on firing pin 101 prevents its ejection in the event that the resisting force is lower than the pressure force. Shaft seal 108 prevents gas flow through the annular clearance between twist shaft 107 and second housing part 109. Twist shaft 107 may be turned directly by the user or indirectly through a mechanism that is part of an application device (not shown).

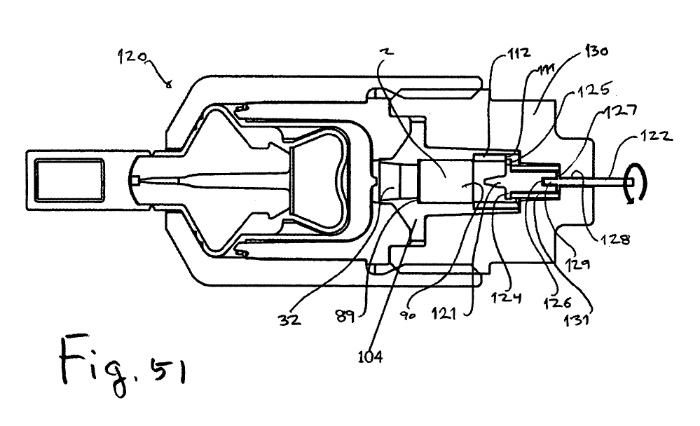

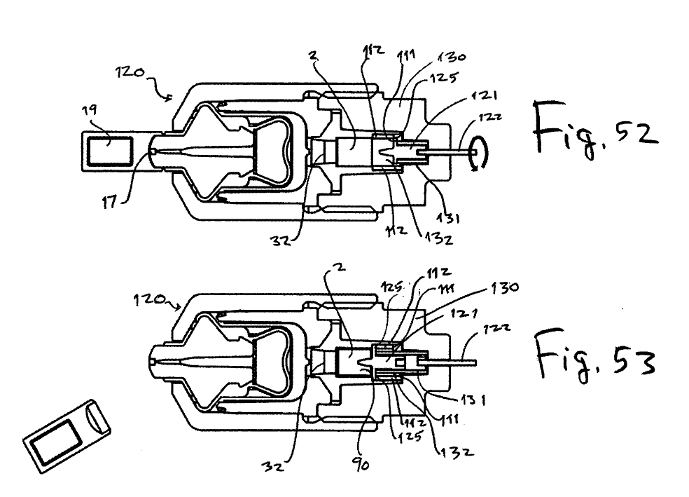

FIGS. 47 and 48 show exploded cross-sectional views of components of device 100 shown by FIGS. 44 through 46 from different points of view. FIGS. 49 and 50 show external views of device 100 shown by FIGS. 44 through 48 from different points of view.

The above-described embodiment 7 offers the advantages of actuation obtained by a rotary motion and when the actuation means reaches a defined angular position.

### Embodiment 8

FIG. 51 shows a sectional view of an injection device 120 having a similar structure and firing pin mechanism as injection device 100 shown in FIG. 44, but wherein firing pin 121 and twist shaft 122 are separate parts, not an integral component, with the result that twist shaft 122 does not move axially with the firing pin, and undergoes rotational motion only.

The ignition means comprises two side open primer 2 positioned within a propellant chamber 123 that supports the outside diameter of primer 2 and locates it axially at the front end 89. Propellant chamber 123 also incorporates support shoulders 111 and release grooves 112. Firing pin 121 includes flange 124 with latch ears 125, and a clutch slot 126. Twist shaft 122 includes clutch blade 129 and seal flange 127, and extends through hole 128 in second housing part 130. Clutch blade 129 on twist shaft 122 engages clutch slot 126 in firing pin 121, such that firing pin 121 rotates when twist shaft 122 is rotated by a torque applied from outside injection device 120. Firing pin 121 is urged towards primer 2 by compressed spring 131 that bears against flange 124. In a first rotational position firing pin 121 is held in this cocked position by the engagement of latch ears 124 with support shoulders 111. In a second rotational position latch ears 124 disengage from support shoulders 111 and align with release grooves 112, freeing firing pin 121 to move axially and make contact with primer 2. Clutch slot 126 in firing pin 121 slides off clutch blade 129 of twist shaft 122 as firing pin 121 moves axially toward primer 2 and twist shaft 122 remains motionless. Preferably, primer 2 contains the full quantity of propellant required for the injection. Optionally, additional propellant may be placed in chamber 32.

FIGS. 52 and 53 illustrate the operating sequence of needleless injection device 120.

FIG. 52 shows needleless injection device 120 prior to the injection. The removable cap 19 is broken off to uncover the sterile injection nozzle 17, and the nozzle is pressed against the patient's skin at the injection site.

FIG. 53 shows needleless injection device 120 during and after the instant of ignition. As a result of turning twist shaft 122 and that is rotationally coupled to firing pin 121, firing pin 121 shifts from a first rotational position in which it is held in a cocked position by the engagement of latch ears 125 with support shoulders 111, and reaches a second rotational position wherein latch ears 125 disengage from support shoulders 111 and align with release grooves 112. Compressed spring 131 is then free to accelerate firing pin 121 so that it strikes and ignites primer 2. The hot products of combustion of primer 2 ignite any optional propellant contained in chamber 32. As described for injection device 60 shown in FIG. 21, the propellant combustion and injection process proceeds. At the same time, hot gas flows from the rear opening 90 of two opening stab primer 2 and into free volume 132. This gas pressure acts on twist shaft 122, and pushes seal flange 127 against second housing part 130 to effect a seal that prevents gas flow through the annular clearance between twist shaft 122 and second housing part 130. Twist shaft 122 may be turned directly by the user or indirectly through a mechanism that is part of an application device (not shown).

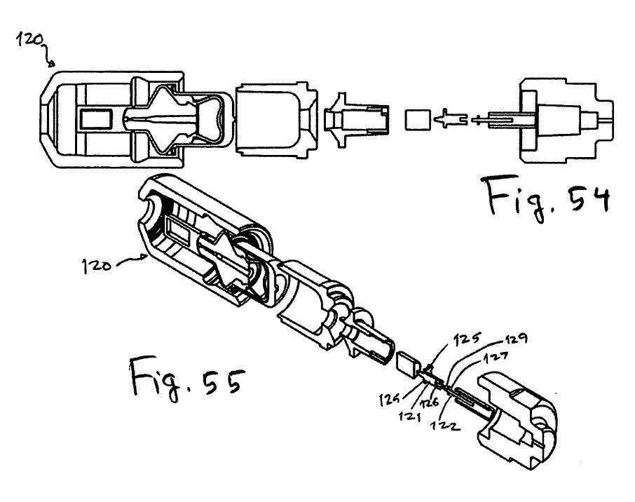

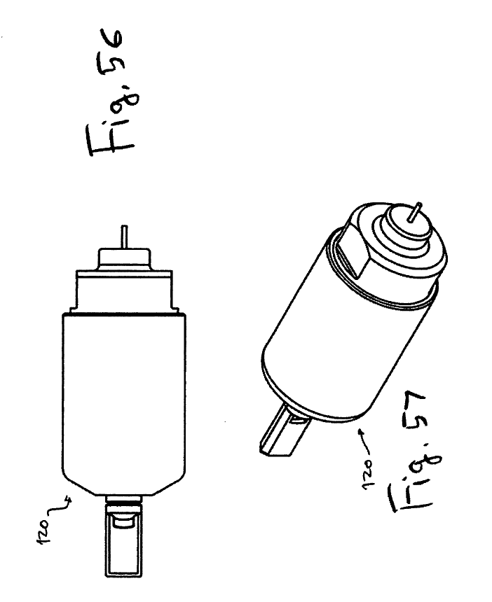

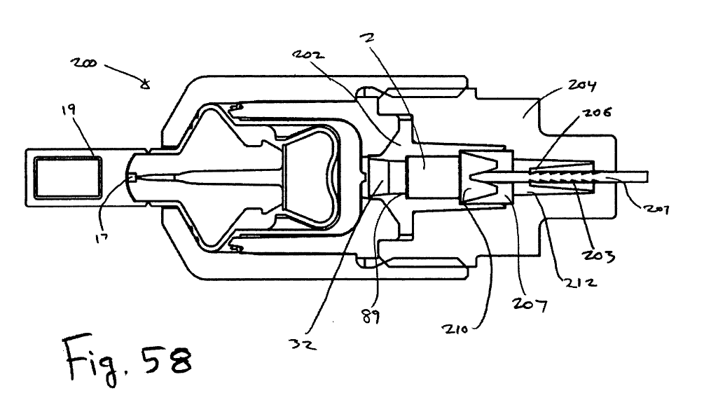

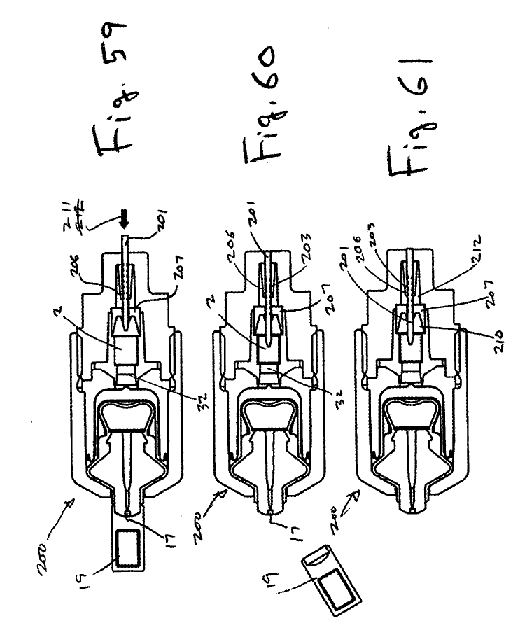

FIGS. 54 and 55 show exploded cross-sectional views of components of device 120 as shown by FIGS. 51 through 53 from different points of view. FIGS. 56 and 57 show external views of device 120 as shown by FIGS. 51 through 55 from different points of view.

The above described embodiment 8 offers the following advantages:

there is no backward motion of the actuating shaft and,

there is a good sealing effect of the propellant chamber at the clutch and this effect is assisted by the pressure generated within the propellant chamber.

### Embodiment 9

FIG. 58 shows a sectional view of an injection device 200 having a similar structure as injection device 1 shown in FIG. 1, but wherein firing pin 5 and cup seal 10 in device 1 are replaced by firing pin 201 and seal 207, the spring and release mechanism are removed from the device, and an externally supplied actuation impact drives firing pin 201 into two side open primer 2.

The ignition means comprises two side open primer 2 positioned within a propellant chamber 202 that supports the outside diameter of primer 2 and locates it axially at the front end 89. Firing pin 201 extends outside second housing 204, and includes ratchet grooves 203. Elastic ratchet fingers 206 engage ratchet grooves 203. Firing pin 201 is free to slide toward primer 2, but is prevented from sliding in the opposite direction by the engagement of ratchet fingers 206 with ratchet grooves 203. Annular volume 212, within second housing 204, surrounds firing pin 201 and ratchet fingers 206. Seal 207 surrounds firing pin 201, and blocks gas flow from free volume 210 to annular volume 212. Preferably, primer 2 contains the full quantity of propellant required for the injection. Optionally, additional propellant may be placed in chamber 32.

FIGS. 59 through 61 illustrate the operating sequence of needleless injection device 200.

FIG. 59 shows needleless injection device 200 prior to the injection. The removable cap 19 is broken off to uncover the sterile injection nozzle 17, and the nozzle is pressed against the patient's skin at the injection site.

FIG. 60 shows needleless injection device 200 at the instant of ignition. An external impact motion 211 accelerates firing pin 201 so that it slides though seal 207 and ratchet fingers 206, and strikes and ignites primer 2. The hot products of combustion of primer 2 ignite any optional propellant contained in chamber 32. The external impact motion 211 may be generated by a known mechanism such as a spring and plunger (not shown).

FIG. 61 shows needleless injection device 200 an instant following ignition. As described for injection device 1 shown in FIG. 1, the propellant combustion and injection process proceeds. At the same time, hot gas flows from the rear opening of two opening stab primer 2 and forces back firing pin 201 through seal 2.07 until ratchet fingers 206 engage ratchet grooves 203 and stop the motion. The effect is to contain the hot gas in free volume 210, and prevent unwanted flow into annular volume 212. Such unwanted flow would result in loss of efficiency through an increase in dead volume, heat losses to solid surfaces, and external leakage. Embodiment 9 is notable in that free volume 210 may be very small.

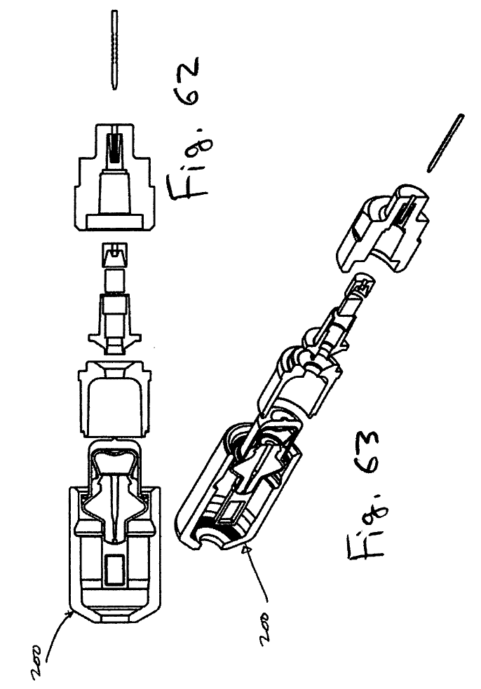

FIGS. 62 and 63 show exploded cross-sectional views of components of device 200 shown by FIGS. 58 through 61 from different points of view.

The above described embodiment 9 offers the following advantages:

since the device does not include any spring for driving the firing pin, there is no contact between the hot gas generated in the propellant chamber and such a spring, and there is no loss of energy that would be caused by such a contact,

only a small volume is available for the expansion of gases generated within the device, and

only a relatively small surface is in contact with combustion gases, i.e., heat loss is very low and thereby loss of injection pressure generated by combustion of the propellant is also very low.

### Aspects Common to the Preceding Embodiments

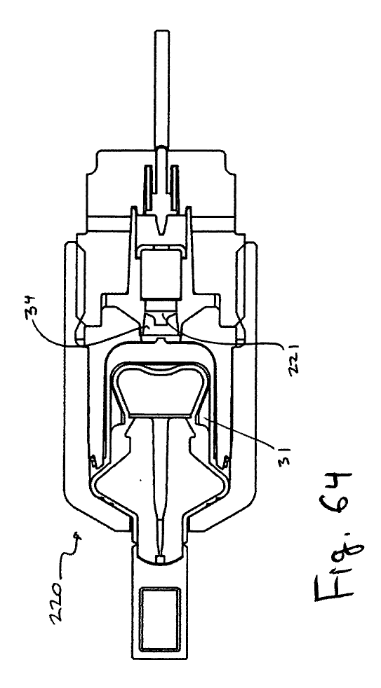

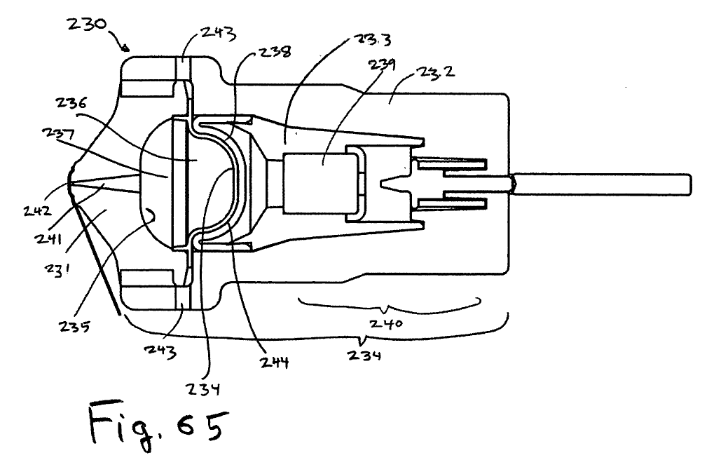

The ignition means described in detail for each of the above embodiments 1 to 9 is suitable for being used with any of the injection device structures described hereinafter with reference to FIGS. 64 to 68.

FIG. 64 shows a sectional view of an injection device 220 having a similar structure and function as injection device 1 shown in FIG. 1, but wherein a breakable membrane 221 separates second chamber 32 from 34, the second zone of first chamber 31. After ignition, breakable membrane 221 confines the gas in second chamber 32 up to predetermined pressure to facilitate combustion, and then bursts to release the gas to carry out the injection. The breakable membrane principle illustrated in injection device 200 is applicable to the ignition and gas generation means described above with reference to FIGS. 1 to 63.

FIG. 65 shows a sectional view of an injection device 230 having similar ignition and gas generation means as injection device 1 shown in FIG. 1, but wherein nozzle body 231 connects to rigid housing 232 to form a shell 246 enclosing volume 233, and further including a first deformable diaphragm 234 which together with a cavity 235 of nozzle body 231 forms a medication chamber 236 suitable for receiving a predetermined amount of a medication 237, and a second deformable diaphragm 238 a portion of which extends around a portion of first deformable diaphragm 234. Second deformable diaphragm 238 and housing 232 form together a chamber 239 that contains a propellant and ignition means 240. Nozzle body 231 has a channel 241 and an orifice 242 at its outer end for ejecting medication 237 out of chamber 236 when a gas pressure generated by propellant and ignition means 240 is applied to second deformable diaphragm 238 and thereby to first deformable diaphragm 234. Vents 243 connect the space 244 between second deformable diaphragm 238 and first deformable diaphragm 234 to the outside of shell 246, such that inadvertent gas leakage through second deformable diaphragm 238 is removed to protect the integrity of first deformable diaphragm 234 and medication 237. Second deformable diaphragm 238 and vents 243 are an optional security measure, and injection device 230 will function with first deformable diaphragm 234 alone. Propellant and ignition means 240 may comprise any of the ignition and gas generation means described above with reference to FIGS. 1 to 63.

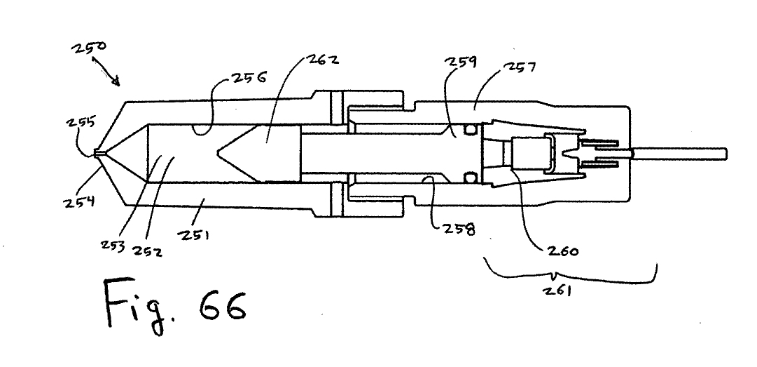

FIG. 66 shows a sectional view of an injection device 250 having similar ignition and gas generation means as injection device 1 shown in FIG. 1, but wherein a rigid medication container 251 having a medication zone 252 for receiving liquid medication 253 defined by a cylindrical bore 256, a nozzle end 254, and a medication piston 262 slidably arranged within cylindrical bore 256. An outlet orifice 255 is in fluidic communication with medication zone 252. A rigid housing 257 connects to medication container 251, and contains a cylindrical bore 258 with a drive piston 259 slidably arranged within and aligned with and contacting medication piston 262, such that motion of drive piston 259 moves medication piston 262 a substantially equal distance. Rigid housing 257 and drive piston 259 together form a propellant zone 260 containing a propellant and ignition means 261, so that upon actuation of propellant and ignition means 261, gas pressure generated by combustion of the propellant causes displacement of drive piston 259, and thereby medication piston 262, which then exerts pressure on liquid medication 253 and ejects it through the outlet orifice 255 of nozzle end 254. Propellant and ignition means 261 may be comprise any of the ignition and gas generation means described above with reference to FIGS. 1 to 63.

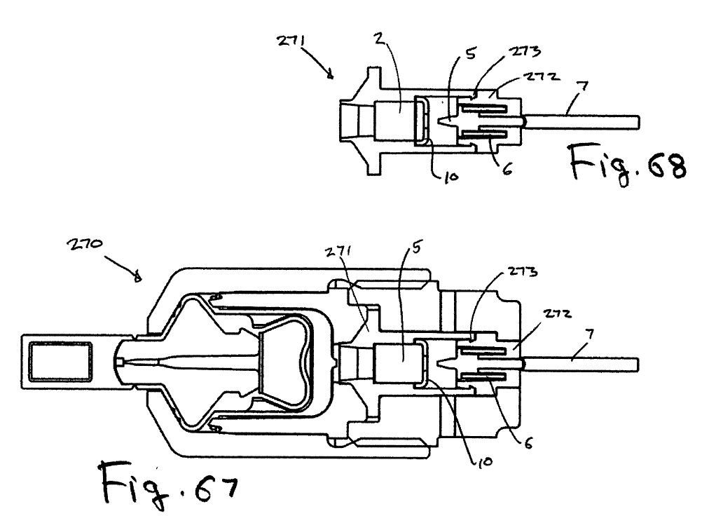

FIG. 67 shows a sectional view of an injection device 270 having similar ignition and gas generation means as injection device 1 shown in FIG. 1, but wherein two-side-open primer 2, firing pin 5, propellant chamber 3, spring 6, release lever 7 and cup seal 10 are combined with a sub-housing 272 by means of a connection 273 to form a separable pyrotechnic module 271 containing gas generation and ignition functions. Module 271 is shown separately in FIG. 68. Functionally, injection device 270 is the same as injection device 1, and the advantage is improved manufacturing logistics. Pyrotechnic module 271 may be manufactured, inspected, stored, and transported separately from the other parts of injection device 270, and added in final assembly. The modular principle illustrated in injection device 270 is applicable to the ignition and gas generation means described above with reference to FIGS. 1 to 66.

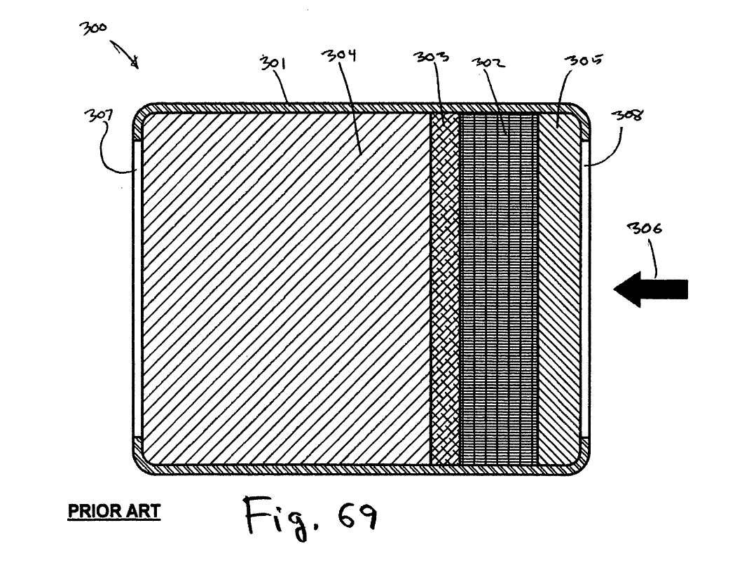

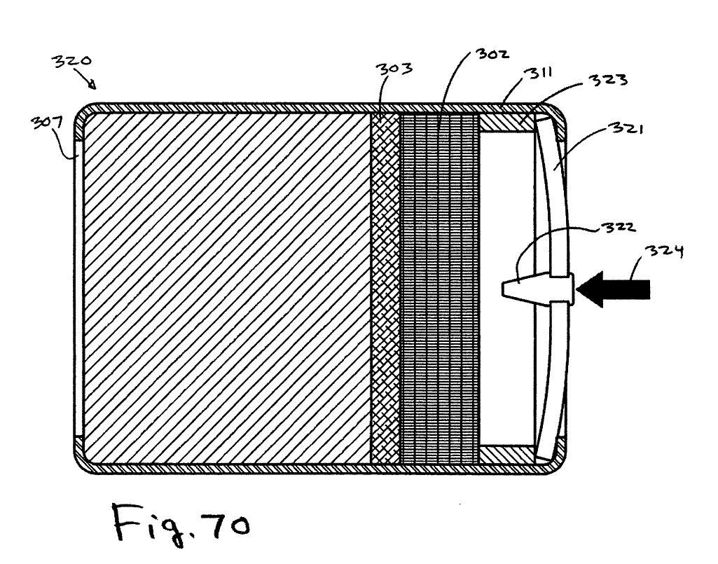

FIG. 69 shows a detailed sectional view of a prior art two side open stab primer 300 such as generally specified as two side open stab primer 5 in FIG. 1 and related figures. A cylindrical metallic outer shell 301 open at the ends 307 and 308 encloses an impact and friction sensitive primer layer 302, a secondary ignition material layer 303 in contact with primer 302, and a propellant layer 304 in contact with layer 303. Layer 305 is paper or foil that protects primer layer 302, but is easily penetrated during ignition. A needle indicated schematically as 306 strikes and penetrates layer 304 and then penetrates and ignites primer layer 302. Secondary ignition material layer 303 in turn ignites and heats and ignites propellant layer 304. Gas is released from both open end 307 and open end 308 of shell 301. One side open style stab primers (not shown) are contained in metal cups similar to shell 301, but closed on end 307, and the gas is released only from the side 308.

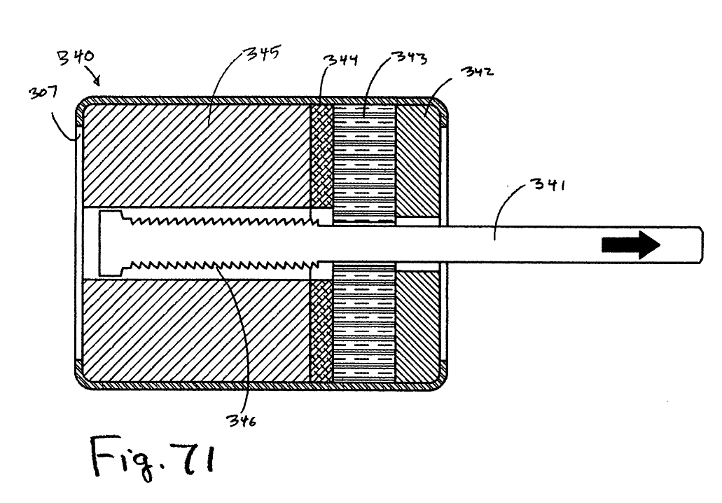

FIG. 70 shows a modular stab igniter 320 similar to the prior art igniter 300 shown in FIG. 69 but wherein an integral bistable spring 321 and firing pin 322 replace paper or foil layer 305 and are enclosed in metallic shell 311 and held in operational relationship to impact and friction sensitive primer layer 302 by spacer ring 323. An external force 324 pushes the center of bistable spring 321 so that it pops through center and drives firing pin 322 into primer layer 302, starting the ignition process. Secondary ignition material layer 303, in turn, heats and ignites propellant layer 304. Gas is released from open end 307 of shell 311. Modular stab igniter 320 may be adapted by one skilled in the art to replace separate stab igniter and firing pin ignition systems in the injection devices described above with reference to FIGS. 1 to 68.

FIG. 71 shows a modular friction igniter 340 similar to the prior art igniter 300 shown in FIG. 69 but wherein a friction firing pin 341 is incorporated. The support disk 342, impact and friction sensitive primer layer 343, secondary ignition material 344 and propellant 345 have central holes to receive friction firing pin 341, with the hole in the primer layer 343 being smallest. Friction firing pin 341 includes serrations 346 that rub strongly against the primer layer 343 as it is pulled out. Alternatively, friction firing pin 341 may be coated with abrasive grit and pyrotechnic material (not shown) in place of serrations 346 to promote friction ignition. When friction firing pin 341 is pulled, frictional interaction between the pin and the primer mix start the ignition process. Secondary ignition material layer 344 in turn ignites and heats and ignites propellant layer 345. Gas is released from open end 307 of shell 301. Friction firing pin 341 may be pulled by a variety of mechanisms operated by the user to trigger the injection. Modular friction igniter 340 is only an example, and other friction primers are possible. In particular, flexible fabric string with an abrasive coating may replace the rigid friction firing pin 341. Modular friction igniter 340 may be adapted by one skilled in the art to replace separate stab igniter and firing pin ignition systems in the injection devices described above with reference to FIGS. 1 to 68.

The ignition means described above with reference to FIGS. 1 to 71 are also suitable for other forms of pyrotechnically powered injection devices, and such applications are included in the scope of the invention.

Although preferred embodiments of the invention have been described using specific terms, such description is for illustrative purposes only, and it is to be understood that changes and variations may be made without departing from the spirit or scope of the following claims.

What is claimed is:

1. A needleless hypodermic injection device for delivering liquid medication contained therein, the device comprising pyrotechnical means for generating within the device a pressure necessary for injecting the medication, and ignition means for igniting a propellant contained in the device, the primer device being part of the ignition means, and comprising a primer material which is adapted to be ignited by friction of the primer material with a mechanical frictional element.

2. The device according to claim 1, wherein the frictional element is an elongated rod, a portion of which is embedded in the primer material, the outer surface of the portion having serrations that cause frictional forces when the rod portion is pulled out of the primer material.

3. The device according to claim 1, wherein the primer device is a stab primer device.

4. The device according to claim 3, wherein the entire amount of propellant in the device is located within the stab primer device.

5. The device according to claim 3, which further comprises an amount of propellant located outside of the stab primer device.

6. The device according to claim 3, wherein the stab primer device contains a firing pin and a bistable spring for driving the firing pin so that it penetrates a primer material contained in the stab primer device.

7. A needleless hypodermic injection device for delivering liquid medication contained therein, the device comprising pyrotechnical means for generating within the device a pressure necessary for injecting the medication, ignition means for igniting a propellant contained in the device, the ignition means comprising
a stab primer device and a firing pin for penetrating a stab primer material stationarily arranged within the stab primer device, the stab primer/material being so positioned with respect to the propellant that when the firing pin penetrates the primer material hot products of combustion of the primer material are generated and these products ignite the propellant.

8. The device according to claim 7, which further comprises
a spring for urging the firing pin towards the primer material, and
a release latch for holding the spring in a loaded position and thereby the firing pin in a cocked position and for releasing the spring and thereby drive the firing pin towards the primer material.

9. The device according to claim 7, wherein the stab primer device comprises a stab primer open on two sides opposite to each other.

10. A needleless hypodermic injection device for delivering liquid medication contained in the device, the device comprising pyrotechnical means for generating within the device a pressure necessary for injecting the medication, and ignition means for igniting a propellant contained in the device, the ignition means comprising
a slidably mounted stab primer device which is open on only one side, the open side being arranged in face of a sharp point of a stationary stab pin,
an impact plunger for driving the stab primer device towards the stab pin so that that pin penetrates into a primer material contained in the stab primer device,
a spring for urging the plunger towards the primer material, and
a release latch for releasably holding the spring and thereby the plunger in a cocked position.

11. The device according to claim 10, which further comprises release means for releasing the release latch.

12. The device according to claim 10, wherein the release means comprises a breakable crimp joint or a breakable rod.

13. The device according to claim 10, wherein the impact plunger comprises a tapered section and a hook for setting the plunger in a cocked position, and wherein the release latch is a release lever for releasing the hook and for thereby releasing the impact plunger from the cocked position.

14. The device according to claim 7, wherein the firing pin ends in a firing pin head located within a closed chamber which seals a space of limited volume located between the firing pin head and one side of the stab primer.

15. The device according to claim 7, which further comprises
a spring for urging the firing pin towards the primer material, and
a release mechanism for holding the spring in a loaded position and thereby the firing pin in a cocked position and for releasing the spring and thereby drive the firing pin towards the primer material.

16. The device according to claim 15, wherein the firing pin, the spring and the release mechanism being located in a closed space into which hot gases generated by ignition of the primer material and the propellant flow.

17. The device according to claim 15, wherein the release mechanism comprises means for rotating the firing pin by means of a torque applied by means located outside the device, the rotating bringing the firing pin from a first angular position where it is in a cocked position to a second angular position where the pin is free to move axially and making contact with the primer material.

18. The device according to claim 15, wherein the release mechanism comprises a shaft adapted to be twisted from a first angular position to a second angular position for releasing the spring, the shaft being in contact with the firing pin in the cocked position thereof, but being mechanically disconnected therefrom so that when the firing pin is released from its cocked position and moves towards the primer material, the shaft does not move with the firing pin.

19. The device according to claim 18, wherein the shaft includes flange means which seals an annular opening around the shaft when pressure pushes the shaft to the rear of the device.

20. The device according to claim 7, wherein the ignition means is an integral part of a preassembled gas generator module.

21. The device according to claim 15, wherein the ignition means is an integral part of a preassembled gas generator module.

22. The device according to claim 7, which further comprises
a bistable spring for urging the firing pin towards the primer material, the bistable spring being adapted to snap at a transition point from a first stable position to a second stable position.

23. The device according to claim 22, which further comprises an actuation screw which when turned in a predetermined position pushes the bistable spring and the firing pin towards the primer material and thereby brings the spring to the transition point where it snaps from the first to the second position, the latter snapping causing the firing pin to penetrate and ignite the primer material.

24. The device according to claim 22, wherein the bistable spring and the firing pin are integral part of the structure of the stab primer.

25. The device according to claim 22, which further comprises an actuation push pin which when axially displaced in a predetermined position pushes the bistable spring and the firing pin towards the primer material and thereby brings the spring to the transition point where it snaps from the first to the second position, the latter snapping causing the firing pin to penetrate and ignite the primer.

26. The device according to claim 22, wherein the bistable spring seals an opening of the stab primer device.

27. The device according to claim 25, which further comprises a venting passage which fluidically connects spaces on opposite sides of the bistable spring and thereby enables gas flow around the bistable spring.

28. The device according to claim 22, wherein the bistable spring has the shape of a disk.

29. The device according to claim 28, wherein the bistable spring comprises vents that equalize pressure on both sides of the disk.

30. The device according to claim 22, wherein the ignition means is an integral part of a preassembled gas generator module.

31. A needleless hypodermic injection device for delivering liquid medication contained therein, the device comprising
(a) a cartridge which contains
(a.1) a medication unit containing the liquid medication,
(a.2) pyrotechnical means for generating within the device a pressure necessary for injecting the medication, and
(a.3) ignition means for igniting a propellant contained in the device,
the ignition means comprising a stab primer device and a firing pin for penetrating a stab primer material stationarily arranged within the device, the stab primer material being so positioned with respect to the propellant that when the firing pin penetrates the primer material hot products of combustion of the primer material are generated and these products ignite the propellant, and
(b) a spring and trigger mechanism for striking the firing pin with such an impact that it strikes and penetrates the primer material, the spring and trigger mechanism being located outside the cartridge.

32. The device according to claim 31, wherein
the firing pin is slidably arranged in a bore of a housing part of the cartridge, a portion of the inner wall of the bore having ratchet fingers,
a part of the firing pin is a shaft a portion of which has a ratchet grooves,
the ratchet fingers and the ratchet grooves being adapted to cooperate with each other to allow motion of the firing pin towards the primer material, but to prevent motion of the firing pin away from the primer material after ignition thereof.

33. The device according to claim 31, wherein the ignition means is an integral part of a preassembled gas generator module.

34. The device according to claim 1, wherein the device further comprising
(a) a housing,
(b) a first chamber within the housing, the first chamber containing a medication unit configured and dimensioned to store a volume of liquid medication to be injected, the medication unit having a first region and a second region that are in liquid communication with each other, the first region being deformable and the second region having an ejection outlet, and
(c) a second chamber within the housing, the second chamber containing a propellant,
the first chamber comprising two zones, a first zone containing the medication unit and a second zone which is in communication with the second chamber, so that upon ignition of the propellant in the second chamber gas generated thereby expands into the second zone of the first chamber, exerts pressure on and deforms the deformable first region of the medication unit and thereby causes ejection of the liquid medication through the ejection outlet.

35. The device according to claim 34, wherein the propellant is contained in a propellant chamber having a wall which has a zone of reduced thickness which upon ignition of the propellant bursts and thereby forms an opening of the wall when gas pressure within the propellant chamber exceeds a predetermined value.

36. The device according to claim 1, wherein the device further comprising
(a) a nozzle body, and
(b) a rigid housing,
the housing having a first open end adapted to receive and be connected with the nozzle body and a second closed end,
the interior of the housing defining a chamber which extends between the open end and the closed end of the housing, the chamber being adapted to receive,
a first deformable diaphragm which together with a cavity of the nozzle body forms a medication chamber suitable for receiving a predetermined amount of a medication, and
a second deformable diaphragm a portion of which extends around a portion of the first deformable diaphragm,
the second deformable diaphragm and the housing forming together a chamber for receiving a propellant as well as means for igniting the propellant, and
the nozzle body having at its outer end an orifice which is the outlet of a channel for ejecting the medication out of the chamber when a gas pressure generated by ignition of the propellant is applied to the second deformable diaphragm and thereby to the first deformable diaphragm.

37. The device according to claim 36, wherein the nozzle body and the housing are connected to form a single structural shell.

38. The device according to claim 36, further comprising venting means for venting of the space comprised between the first deformable diaphragm and the second deformable diaphragm.

39. The device according to claim 36, without the second deformable diaphragm.

40. The device according to claim 1, wherein the device further comprising
(a) a rigid medication container having a medication zone for receiving the liquid medication,
(b) a nozzle in fluidic communication with the medication zone, the nozzle having an outlet orifice,
(c) a propellant zone where the propellant is located within the device,
(d) a channel that fluidically connects the propellant zone with the medication zone, and
(e) piston means slidably arranged within the channel, so that upon ignition of the propellant gas pressure generated by combustion of the propellant causes displacement of the piston means which then exert pressure on the liquid medication and eject it through the outlet orifice of the nozzle.
